SEQUENCE LISTING (3 sequences)

SEQ ID NO: 1 - DNA, 22 bp, Artificial Sequence
  Synthetic polynucleotide
tggagcatgtggtttaattcga

SEQ ID NO: 2 - DNA, 19 bp, Artificial Sequence
  Synthetic polynucleotide
tgcgggacttaacccaaca

SEQ ID NO: 3 - DNA, 23 bp, Artificial Sequence
  Synthetic polynucleotide
  misc_feature (1)..(1): Cy5
  misc_feature (23)..(23): BHQ3
cacgagctgacgacarccatgca

(19) United States
(12) Patent Application Publication
(10) Pub. No.: US 2022/0160712 A1
(43) Pub. Date: May 26, 2022

(54) METHODS OF TREATING RHEUMATOID ARTHRITIS AND FOR PREDICTING THE RESPONSE TO METHOTREXATE

(71) Applicants: CHAN ZUCKERBERG BIOHUB, INC., San Francisco, CA (US); THE REGENTS OF THE UNIVERSITY OF CALIFORNIA, Oakland, CA (US)

(72) Inventors: Peter Turnbaugh, Oakland, CA (US); Renuka R. Nayak, Oakland, CA (US)

(21) Appl. No.: 17/198,023

(22) Filed: Mar. 10, 2021

Related U.S. Application Data
(60) Provisional application No. 62/988,152, filed on Mar. 11, 2020.

(51) Int. Cl. A61K 31/519; A61K 31/192; A61K 31/573; A61K 31/42; A61K 31/4706; A61K 31/655; A61K 31/52; A61K 31/65; A61K 38/17; A61K 39/395; A61P 29/00; C12Q 1/689; C12Q 1/6883

(52) U.S. Cl. CPC A61K 31/519; C12Q 2600/158; A61K 31/573; A61K 31/42; A61K 31/4706; A61K 31/655; A61K 31/52; A61K 31/65; A61K 38/1774; A61K 39/3955; A61P 29/00; C12Q 1/689; C12Q 1/6883; C12Q 2600/106; A61K 31/192

(57) ABSTRACT

The present disclosure provides materials and methods for reducing inflammation in the gut and/or joints of a patient, and optionally treating rheumatoid arthritis (RA) and other inflammatory or autoimmune diseases, in a patient. The present disclosure provides such methods based, in part, on the susceptibility of the patient's gut microbiome to methotrexate (MTX) as described herein.

### CROSS-REFERENCE TO RELATED APPLICATIONS

This application claims priority to U.S. Provisional Patent Application No. 62/988,152 filed Mar. 11, 2020.

### STATEMENT REGARDING FEDERALLY SPONSORED RESEARCH

This invention was made with government support under grants K08 AR073930, R01 HL122593, R01 AR074500 and R03 AR072182 awarded by The National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55261p1_Seqlisting.txt”, which was created on Mar. 10, 2020 and is 1,031 bytes in size. The subject matter of the Sequence Listing is incorporated herein in its entirety by reference.

### FIELD

The present disclosure relates generally to methods of reducing inflammation in the gut and/or joints of a patient, and optionally treating rheumatoid arthritis (RA) and other inflammatory or autoimmune diseases, in a patient. The present disclosure provides such methods based, in part, on the susceptibility of the patient's gut microbiome to methotrexate (MTX) as described herein.

BACKGROUND Rheumatoid arthritis is an autoimmune disease of unknown etiology that leads to inflammation and destruction of joints as well as other organs. Nearly all newly diagnosed RA patients are initiated on methotrexate; however, only ˜33% of patients demonstrate a robust response without escalation of therapy (Weinblatt, M. E., et al., J Rheumatol 25, 238-242 (1998), and Romao, V. C., BMC Med 11, 17 (2013)). In cancer cells, MTX targets dihydrofolate reductase (DHFR) (Bleyer, W. A., Cancer 41, 36-51 (1978)), an enzyme that converts folic acid into tetrahydrofolate (THF), which is required to produce purines and pyrimidines, as well as proteins and lipids (Cronstein, B. N., Arthritis Rheum 39, 1951-1960 (1996). Yet despite more than 60 years of use for RA therapy, the mechanism of action remains unclear and is thought to involve additional mechanisms beyond DHFR due to the use of low doses (Visser, K. & van der Heijde, D., Ann Rheum Dis 68, 1094-1099 (2009)) and the co-administration of folic acid without an attendant loss in efficacy (Cronstein, B. N., Arthritis Rheum 39, 1951-1960 (1996)). Non-antibiotic drugs such as MTX profoundly alter the human microbiome (Falony, G., et al., Science 352, 560-564 (2016), and Maier, L., et al., Nature (2018)); however, the downstream consequences for treatment outcomes remain unclear.

### SUMMARY OF THE INVENTION

In various aspects, the present disclosure provides methods for treating inflammation in the gut and/or joints of a subject, e.g., associated with rheumatoid arthritis (RA) or other diseases and disorders. In one aspect, a method of treating rheumatoid arthritis (RA) in a subject is provided comprising the steps of: a. obtaining a first microbiome sample from the subject, wherein said subject has not received methotrexate, and determining the abundance of at least one bacterial phyla in said sample; b. obtaining a second microbiome sample from the subject, wherein said subject has received at least one dose of methotrexate, and determining the abundance of at least one bacterial phyla in said sample; c. comparing the abundance of step (a) with the abundance of step (b); and d. administering methotrexate (MTX) if the abundance of step (b) is less than the abundance of step (a), or administering a medication other than MTX if the abundance of step (b) is equal to or more than the abundance of step (a). In one aspect, the abundance is measured by 16S RNA copy number per gram of sample. In another aspect, the subject is human.

In other aspects, the present disclosure provides an aforementioned method wherein said subject received 2, 3, 4 or 5 doses of MTX prior to obtaining the second sample. In some aspects, the subject received said doses for 1, 2, 3, 4, or more weeks prior to obtaining said second sample.

In still other aspects, the present disclosure provides an aforementioned method wherein the sample is selected from the group consisting of a fecal sample, a biopsy, and a noninvasive capsule endoscopy sample.

In other aspects, the present disclosure provides an aforementioned method wherein the abundance of 2, 3, 4, 5 or more phyla of bacteria is determined. In some aspects, the bacterial phyla is selected from the group consisting of Bacteroidetes, Firmicutes, Actinobacteria, Proteobacteria, Fusobacteria and Verrucomicrobia. In one aspect, the phylum is Bacteroidetes. In some aspects, the Bacteroidetes comprises one or more of Sphingobacteriia, Bacteroidales, Bacteroidaceae, Tannerellaceae, Rikenellaceae, Prevotellaceae, Bacteroides, Tannerella, Parabacteroides, Alistipes, Prevotella, Bacteroides fragilis, Bacteroides vulgatus, Bacteroides uniformis, Parabacteroides disasonis, Alistipes finegoldii, and Prevotella spp.

In still other aspects, the present disclosure provides an aforementioned method wherein the bacterial phyla comprises:

(a) one or more members of a class selected from the group consisting of Negativicutes, Bacteroidia, Clostridia, Betaproteobacteria, Erysipelotrichia, Actinobacteria, Deltaproteobacteria, Verrucomicrobiae, Coriobacteriia, and Bacilli;

(b) one or more members of an order selected from the group consisting of Selenomonadales, Bacteroidales, Clostridiales, Erysipelotrichales, Coriobacteriales, Desulfovibrionales, Verrucomicrobiales, Burkholderiales, Lactobacillales, and Bifidobacteriales;

(c) one or more members of a family selected from the group consisting of Acidaminococcaceae, Porphyromonadaceae, Lachnospiraceae, Ruminococcaceae, Bacteroidaceae, Eubacteriaceae, Erysipelotrichaceae, Coriobacteriaceae, Desulfovibrionaceae, Clostridiales Incertae Sedis XIII, Clostridiaceae Prevotellaceae, Verrucomicrobiaceae, Sutterellaceae, Defluviitaleaceae, Streptococcaceae, Bifidobacteriaceae, Clostridiaceae, and Leuconostocaceae;

(d) one or more members of a genus selected from the group consisting of Phascolarctobacterium, Barnesiella, Dorea, Blautia, Clostridium XlVa, Clostridium XlVb, Anaerorhabdus, Eubacterium, Bacteroides, Eggerthia, Collinsella, Flavonifractor, Ruminococcus, Gordonibacter, Bilophila, Anaerofustis, Mogibacterium, Coprococcus, Oscillibacter, Clostridium sensu stricto, Lachnospiraceae UCG-008, Lachnoclostridium, Eubacterium fissicatena group, Dielma, Marvinbryantia, Subdoligranulum, Lachnospiraceae NK3A20 group, Prevotella, Akkermansia, Odoribacter, Clostridium XVIII, Ruminococcus2, Parasutterella, Clostridium IV, Defluviitalea, Streptococcus, Bifidobacterium, Butyricicoccus, Clostridium sensu stricto, and Weissella; and

(e) one or more members of a species selected from the group consisting of Phascolarctobacterium faecium, Blautia hansenii/producta, Clostridium XlVa bolteae/clostridioforme, Clostridium XlVa scindens, Bacteroides uniformis, Clostridium XlVa oroticum, Bacteroides ovatus, Bacteroides caccae, Bacteroides cellulosilyticus/timonensis, Bacteroides intestinalis, Collinsella aerofaciens, Blautia faecis, Anaerofustis stercorihominis, Clostridium sensu stricto celatum/disporicum, Lachnospiraceae UCG-008 uncultured organism, Bacteroides thetaiotaomicron, Marvinbryantia metagenome, Lachnospiraceae NK3A20 group uncultured bacterium, Akkermansia muciniphila, Bacteroides dorei/vulgatus, Bacteroides massiliensis, Coprococcus comes, Blautia obeum, Bacteroides faecichinchillae/faecis/thetaiotaomicron, Clostridium XlVa glycyrrhizinilyticum, Parasutterella excrementihominis, Streptococcus anginosus subsp. anginosus, Bacteroides uniformis, Bacteroides thetaiotaomicron, and Butyricicoccus uncultured bacterium.

In yet other aspects, the present disclosure provides an aforementioned method wherein the MTX is selected from the group consisting of RASUVO®, TREXALL®, OTREXUP®, Methotrexate LPF Sodium, XATMEP®, JAMP-Methotrexate, METOJECT® and PMS-Methotrexate. In some aspects, the MTX is administered by a route selected from the group consisting of subcutaneous injection, intravenous injection, and oral tablet.

In other aspects, the present disclosure provides an aforementioned method wherein the medication is selected from the group consisting of nonsteroidal anti-inflammatory drugs (NSAIDs), steroids, disease-modifying antirheumatic drugs (DMARDs), and biological agents. In some aspects, the NSAID is selected from the group consisting of ibuprofen and naproxen sodium. In other aspects, the steroid is selected from the group consisting of prednisone, prednisolone, methylprednisolone, dexamethasone, hydrocortisone, triamcinolone, and betamethasone. In some aspects, the DMARD is selected from the group consisting of leflunomide (Arava), hydroxychloroquine (PLAQUENIL®), sulfasalazine (AZULFIDINE®), azathioprine (IMURAN®) and minocycline. In still other aspects, the biological agent is selected from the group consisting of abatacept (ORENCIA®), adalimumab (HUMIRA®), anakinra (KINERET®), baricitinib (OLUMIANT®), certolizumab (CIMZIA®), etanercept (ENBREL®), golimumab (SIMPONI®), infliximab (REMICADE®), rituximab (RITUXAN®), sarilumab (KEVZARA®), tocilizumab (ACTEMRA®), tofacitinib (XELJANZ®), and upadacitinib (RINVOQ®).

In another aspect, the present disclosure provides a method of reducing inflammation in the gut and/or joints of a subject comprising the steps of: a. obtaining a sample from the subject; b. determining the abundance of at least one bacterial phylum in the sample; c. comparing the abundance of said at least one bacterial phylum to a threshold amount; and d. administering methotrexate (MTX) if the abundance of said at least one bacterial phylum is less than said threshold amount, or administering a medication other than MTX if the abundance of said at least one bacterial phylum is more than said threshold amount.

In some aspects, the subject is suffering from RA and/or inflammatory bowel disease (IBD). In another aspect, the IBD is selected from the group consisting of Crohn's disease or ulcerative colitis.

In other aspects, the present disclosure provides an a method of treating rheumatoid arthritis (RA) in a subject comprising the steps of: a. obtaining a microbiome sample from the subject; b. determining the abundance of at least one bacterial phyla in the sample; c. comparing the abundance of said at least one bacterial phyla to a threshold amount; and d. administering methotrexate (MTX) if the abundance of said at least one bacterial phyla is less than said threshold amount, or administering a medication other than MTX if the abundance of said at least one bacterial phyla is more than said threshold amount.

### BRIEF DESCRIPTION OF THE DRAWINGS

FIGS. 1A-1I show that MTX alters the gut microbiotas of humanized mice within days. FIG. 1A: qPCR was used to assess bacterial colonization level (16S copy number per gram stool) at baseline and after 4 days of MTX treatment. Treatments: “-”, vehicle; “L”, low-dose MTX (1 mg/kg); “H” high-dose MTX (50 mg/kg). n=3 mice/group, 1 cage/group. FIG. 1B: Number of amplicon sequence variants (ASVs) detected using 16S rRNA gene sequencing. Statistical results of Kruskal-Wallis test reported. FIG. 1C: Principal components analysis (PCA) of Euclidean distances using clr-transformed values from longitudinal stool samples. ANOSIM results are shown. Shifts in panel C were confirmed by PERMANOVA also (R2=0.68, p=0.001; Day 4). FIG. 1D: Bacteroidetes significantly decreased with high-dose MTX treatment relative to vehicle controls (DESeq padj=0.001, Day 4). Lines depict the mean for each group. FIG. 1E: Differentially abundant ASVs for each dose relative to vehicle (DESeq padj<0.01, Day 4). FIG. 1F: All of the ASVs differentially abundant in response to both doses showed consistent directionality (p=1.02×10-7, Hypergeometric test). Gray scale indicate log2 fold changes (log2FC) (Day 4, relative to vehicle). FIG. 1G-H: Principal components analysis (PCA) of Euclidean distances using clr-transformed values endpoint cecal (G) and colon contents (H). ANOSIM results are shown for each graph. FIG. 1I:UpSet plot of differentially abundant ASVs when comparing high-dose MTX vs. vehicle in endpoint cecum, colon and fecal samples (Day 4).

FIGS. 2A-2E show that MTX directly inhibits the growth of human gut bacterial isolates. FIG. 2A: A diverse panel of 45 isolates were incubated with varying concentrations of MTX and the minimal inhibitory concentration (MIC) was measured. A maximum likelihood phylogenetic tree using full-length 16S rRNA gene sequences for each organism was constructed, with bootstrap values greater than 70/100 iterations indicated by circles at branch points. The tree shows 43 of the isolates (2 additional E. lenta strains were tested but only one of each species was included in the tree). FIG. 2B: The MICs of various isolates spanning 4 major phyla. Bacteroidetes were compared to other phyla (Wilcoxon rank-sum test). Boxplot top and bottom hinges correspond to the first and third quartiles, respectively, and horizontal lines denote the median. FIG. 2C: The cumulative percentage of 45 tested human gut bacterial isolates that have MICs at or lower than a given concentration of MTX. FIG. 2D: Growth curves of 4 representative isolates incubated with 10 concentrations of MTX. Each isolate was tested in duplicate and averages are shown. FIG. 2E: Carrying capacity, growth rate, and lag phase parameters were affected in a dose-dependent manner by MTX among a significant proportion of bacteria.

FIGS. 3A-3D show the Impact of MTX on human gut bacterial transcriptomes and metabolomes. FIG. 3A: A variable number of transcripts, as determined by RNA-seq, were differentially expressed (padj<0.2, DESeq) upon 30 minutes of MTX 100 μg/ml (compared to vehicle control) in 4 bacterial isolates with varying sensitivity to the growth inhibitory effects of the drug (n=3 per treatment). Clostridium asparagiforme was also treated for 4 and 20 hours. FIG. 3B: A heatmap of KEGG pathway enrichments (padj<0.05, BH corrected) of differentially expressed genes (padj<0.2, DESeq) in bacterial isolates treated with 100 μg/ml of MTX vs. vehicle control (n=3 per treatment / isolate). Gray scale represent log10 adjusted p-value. FIG. 3C: Pathways enriched among metabolite features exhibiting a significant interaction between bacterial isolate and MTX treatment in the supernatants of B. theta and C. asparagiforme cultures exposed to MTX (100 μg/ml for 24 hours) as assessed by mummichog and gene set enrichment analysis (GSEA) (n=2 replicates / isolate for DMSO vs. n=4 replicates/isolate for MTX). Dark circles are significant, and circle size indicates number of metabolite features. FIG. 3D: Multiple enzymes involved in purine metabolism (Pedley, A. M., and Benkovic, S. J. (2017), Trends Biochem Sci 42, 141-154) were affected at some point during the time course study that was performed on C. asparagiforme. A 3-box heatmap of log2 fold change values at 30 minutes, 4 hours, and 20 hours is shown above each enzyme, with asterisks indicating padj values. Asterisks indicate padj: *<0.2, **<0.00001, ***<0.000001. Four metabolites were detected with high confidence by untargeted metabolomics at 24 hours of treatment (n=2 DMSO and n=6 for MTX): AMP, adenine, hypoxanthine and guanine. Levels of AMP were decreased relative to vehicle control, and the rest were increased, but these differences were not statistically significant (Wilcoxon rank sum). DHF, dihydrofolate; DHFR, dihydrofolate reductase; THF, tetrahydrofolate; 10-fTHF, 10-formyl-tetrahydrofolate; PPRP, phosphoribosylpyrophosphate; PPAT, PPRP amidotransferase; PRA, 5-phosphoribosylamine; GART, trifunctional enzyme consisting of phosphoribosylglycinamide synthetase (GARS), phosphoribosylglycinamide formyltransferase (GAR Tfase), and phosphoribosylaminoimidazole synthetase (AIRS); GAR, glycineamide ribonucleotide; FGAR, N-formylglycinamide ribonucleotide; FGAMS, phosphoribosyl formylglycinamidine synthase; FGAM, N-formylglycinamidine ribonucleotide; AIR, aminoimidazole ribonucleotide; PAICS, bifunctional enzyme consisting of phosphoribosyl aminoimidazole carboxylase (CAIRS) and phosphoribosyl aminoimidazole succinocarboxamide synthetase (SAICARS); CAIR, 5′-phosphoribosyl-4-carboxy-5-aminoimidazole; SAICAR, N-succinocarboxyamide-5-aminoimidazole ribonucleotide; ADSL, adenylosuccinate lyase; AICAR, aminoimidazole-4-carboxamide ribonucleotide; ATIC, bifunctional enzyme consisting of 5-aminoimidazole-4-carboxamide ribonucleotide formyltransferase (AICAR Tfase) and IMP cyclohydrolase (IMPCH); FAICAR, 5-formamidoimidazole-4-carboxamide ribotide; IMP, inosine 5′-monophosphate; ADSS, adenylosuccinate synthase; AMPS, adenylosuccinate; AMP, adenosine monophosphate; APRT, adenine phosphoribosyltransferase; HPRT, hypoxanthine-guanine phosphoribosyltransferase; IMPDH, IMP dehydrogenase; XMP, xanthosine monophosphate; GMPS, GMP synthase; GMP, guanosine 5′-monophosphate.

FIGS. 4A-4F show that Folic acid and leucovorin partially rescue the effects of MTX. FIG. 4A-B: Growth curves (A) and area under the growth curve (AUC) (B) of 4 isolates incubated with MTX (61 μM, 28 μg/ml) and varying concentrations of folic acid. Growth control curves are shown for comparison. FIG. 4C: Slope estimates ((3) and p-values from linear regressions estimating the effects of folic acid on AUC for 4 isolates incubated with 9 concentrations of MTX and 7 concentrations of folic acid. FIG. 4D-E: Growth curves (D) and area under the growth curve (AUC) (E) of 4 isolates incubated with MTX (61 μM, 28 μg/ml) and varying concentrations of leucovorin. Growth control curves are shown for comparison. FIG. 4F: Slope estimates ((3) and p-values from linear regressions estimating the effects of leucovorin on AUC for 4 isolates incubated with 9 concentrations of MTX and 7 concentrations of leucovorin. FIG. 4C, F: Panel backgrounds are grayed depending on whether the rescue agent significantly (p<0.05; linear regression) enhances or impairs growth, respectively.

FIGS. 5A-5N show that MTX alters the human gut microbiota ex vivo and in treatment responsive patients. FIG. 5A: Growth of fecal suspensions from treatment-naive RA patients (n=30, 4 replicates/treatment) in the presence of MTX 100 μg/ml or DMSO. The average growth curves of 30 patients are shown. Shaded areas represent ±SEM. Carrying capacity was reduced (p=1.9×10-6, paired Student's t-test). FIG. 5B-E: PCA of Euclidean distances using clr-transformed values from ex vivo patient samples (MTX-R) treated with MTX vs. vehicle control at 0 and 24 hours. ANOSIM testing was performed comparing MTX to vehicle control at the different time points. FIG. 5F-G: Normalized abundances (clr) of Bacteroidetes (F) and Actinobacteria (G) phylum levels in 4 ex vivo microbial communities treated with MTX (100 μg/ml) or DMSO for 24 hours (n=4 replicates/treatment per patient) (padj<0.01, DESeq comparing DMSO vs. MTX). FIG. 5H: Phylogenetic tree of 20 ASVs that are differentially abundant (padj<0.01, DESeq) with MTX treatment ex vivo in 4 patient samples. FIG. 5I: Fecal samples from 23 RA patients were subjected to 16S sequencing before treatment with MTX and 1 month after treatment. Relative abundance of Bacteroidetes is shown for each individual (blue, decreased; yellow, increased). FIG. 5J-N: Change in relative abundance of 5 major phyla with MTX treatment in responders compared to non-responders (Wilcoxon rank-sum).

FIGS. 6A-6B show that MTX-altered microbiotas dampen host immune activation. FIG. 6A: Heatmap showing the relative percentage of cell populations in post-MTX recipient mice under unchallenged and challenged conditions (normalized to the pre-MTX recipient group for each condition) (n=20-22 mice/donor sample, 2-5 cages/treatment group). Populations that significantly differed (p<0.05; linear mixed effects model) are indicated by an asterisk and the fold change percentage in post-MTX recipient mice is indicated (i.e., post-MTX levels divided by pre-MTX levels). FIG. 6B: Immune cell populations in mice in 3 donor experiments. Each bar indicates the mean fold change percentage (relative to pre-MTX recipient mice for each condition). P-values from linear mixed effects models are reported.

FIGS. 7A-7E show that Gut microbial taxa altered by MTX are associated with immune cell populations. FIG. 7A: Of 12 ASVs that were modulated by MTX in prior experiments, 11 ASVs exhibit 25 significant correlations with immune markers in the spleen and colon of Donor 2 pre-MTX and post-MTX recipient mice. Four representative ASV-immunocyte correlations are boxed and underlying data are shown in panels B-E. Asterisks denote significant correlations. ASVs that were previously shown to be downregulated or upregulated by MTX are shaded. FIG. 7B-E: The left and right panels depict data from two separate experiments. The left panel depicts ASV abundances from individually housed mice treated with high-dose MTX. The right panel depicts correlations between ASV abundance and immune cell percentages in mice that were transplanted with pre-MTX or post-MTX stool samples from patients treated with MTX. Mice in both of these experiments were colonized by the same RA patient donor (Donor 2). FIG. 7B: ASV23 (Bacteroides faecichinchillae/faecis/finegoldii/thetaiotaomicron) abundance is reduced by MTX (left panel) and is positively associated with T cell activation markers (CD44+CD69+CD4+) in the spleen (right panel). FIG. 7C: ASV296 (Lachnoclostridium) abundance is reduced by MTX (left panel) and is positively associated with T cell activation markers (CD44+CD69+CD4+) in the spleen (right panel). FIG. 7D: Dielma fastidiosa (ASV72) abundance is decreased by MTX (left panel) and this ASV is positively correlated with T cell activation markers in the spleen (right panel). FIG. 7E: ASV908 (Paraprevotella) abundance is increased by MTX (left panel) and this ASV is negatively correlated with myeloid cell markers in the colon (right panel).

### DETAILED DESCRIPTION

The present disclosure demonstrates that methotrexate (MTX), the first-line therapy for rheumatoid arthritis (RA), is sufficient to affect the growth of human gut bacteria in isolation, complex communities, and within the gastrointestinal tract. As in eukaryotes, MTX targets bacterial purine and pyrimidine biosynthesis, selecting against the growth of members of the Bacteroidetes phylum among other sensitive strains. As described herein, MTX-induced shifts in the gut microbiota were associated with clinical response, consistent with human to mouse microbiome transplantations which led to reduced immune activation in mice receiving post-treatment gut microbiomes. These results indicate non-antibiotic immunomodulatory drugs may act in part through off-target effects on the gut microbiota, while providing a critical first step towards explaining long-standing differences in drug response between patients.

In various aspects, a method of altering bacterial abundance of microbiota in digestive organs of a subject in need thereof are provided, comprising administering to the subject a composition comprising methotrexate. In some embodiments, the digestive organs include the gut, intestines and digestive track of the subject.

In other embodiments, a method for enhancing a therapy of a disease or condition in a human or animal patient is provided, the method comprises selective killing or reducing growth of a target bacterial sub-population of a microbiota using a therapeutic agent, for example methotrexate. For example, while the present disclosure describes that methotrexate treatment itself can reduce the abundance of certain bacterial microbiome subpopulations, it is also contemplated that other treatments can be used for selective killing or reducing growth of the bacterial microbiome subpopulations, followed by methotrexate treatment. For example, U.S. Pat. No. 10,300,138, incorporated by reference in its entirety herein, discloses methods of modulating immune cells in a patient by altering microbiota of the patient, and methods of modulating treatments or therapies in a subject organism by altering microbiota of the subject.

As used herein, the term “microbiome” refers to the community of organisms and genetic material of all microbes—bacteria, fungi, protozoa and viruses—that live on and inside the human body. The bacteria in the microbiome help digest our food, regulate our immune system, protect against other bacteria that cause disease, and produce vitamins, including B vitamins B12, thiamine and riboflavin, and vitamin K, which is needed for blood coagulation. As used herein, then, the term “gut microbiome” refers to the microorganisms and genetic material therein that live in the digestive organs which include the gut, intestines and digestive track.

The microbiome is essential for human development, immunity and nutrition. The bacteria living in and on us are not invaders but beneficial colonizers. Autoimmune diseases such as diabetes, rheumatoid arthritis, muscular dystrophy, multiple sclerosis, and fibromyalgia are associated with dysfunction in the microbiome. Disease-causing microbes accumulate over time, changing gene activity and metabolic processes and resulting in an abnormal immune response against substances and tissues normally present in the body. Autoimmune diseases appear to be passed in families not by DNA inheritance but by inheriting the family's microbiome. A person's microbiome may influence their susceptibility to infectious diseases and contribute to chronic illnesses of the gastrointestinal system like Crohn's disease and irritable bowel syndrome.

Each individual is provided with a unique gut microbiota profile that plays many specific functions in host nutrient metabolism, maintenance of structural integrity of the gut mucosal barrier, immunomodulation, and protection against pathogens. Gut microbiota are composed of different bacteria species taxonomically classified by genus, family, order, and phyla. Each human's gut microbiota are shaped in early life as their composition depends on infant transitions (birth gestational date, type of delivery, methods of milk feeding, weaning period) and external factors such as antibiotic use. These personal and healthy core native microbiota remain relatively stable in adulthood but differ between individuals due to enterotypes, body mass index (BMI) level, exercise frequency, lifestyle, and cultural and dietary habits. Accordingly, there is not a unique optimal gut microbiota composition since it is different for each individual. However, a healthy host-microorganism balance must be respected in order to optimally perform metabolic and immune functions and prevent disease development. Dysbiosis of gut microbiota is associated not only with intestinal disorders but also with numerous extra-intestinal diseases such as metabolic and neurological disorders. Understanding the cause or consequence of these gut microbiota balances in health and disease and how to maintain or restore a healthy gut microbiota composition should be useful in developing promising therapeutic interventions.

In various embodiments, the methods described herein can be used to treat a disease or alleviate the symptoms in a mammal, e.g, a human, man or woman, or male child or female child, or a human infant (e.g., no more than 1, 2, 3 or 4 years of age). Samples from such subjects may be obtained using a variety of techniques known in the art including, but limited to, collection of fecal sample, a biopsy, and a noninvasive capsule endoscopy sample.

As discussed in Rinninella et al., (Rinninella, E., et al., Microorganisms, 7(1):14 (2019); incorporated by reference in its entirety herein) Gut microbiota are composed of several species of microorganisms, including bacteria, yeast, and viruses. Taxonomically, bacteria are classified according to phyla, classes, orders, families, genera, and species. Only a few phyla are represented, accounting for more than 160 species. The dominant gut microbial phyla are Firmicutes, Bacteroidetes, Actinobacteria, Proteobacteria, Fusobacteria, and Verrucomicrobia, with the two phyla Firmicutes and Bacteroidetes representing 90% of gut microbiota. The Firmicutes phylum is composed of more than 200 different genera such as Lactobacillus, Bacillus, Clostridium, Enterococcus, and Ruminicoccus. Clostridium genera represent 95% of the Firmicutes phyla. Bacteroidetes consists of predominant genera such as Bacteroides and Prevotella. The Actinobacteria phylum is proportionally less abundant and mainly represented by the Bifidobacterium genus.

As used herein, the term “phyla” or “phylum” refer to the major lineages of the domain Bacteria. As described herein, abundances of various taxonomic levels are decreased in response to MTX including phylum, class, order, family, genus and species. Exemplary members of each group are provided herein:

Exemplary bacteria that are sensitive to MTX (e.g., decrease in abundance in response to MTX):

Phyla: Firmicutes, Bacteroidetes, Proteobacteria, Actinobacteria, and Verrucomicrobia.

Class: Negativicutes, Bacteroidia, Clostridia, Betaproteobacteria, Erysipelotrichia, Actinobacteria, Deltaproteobacteria, Verrucomicrobiae, Coriobacteriia, and Bacilli.

Order: Selenomonadales, Bacteroidales, Clostridiales, Erysipelotrichales, Coriobacteriales, Desulfovibrionales, Verrucomicrobiales, Burkholderiales, Lactobacillales, and Bifidobacteriales.

Family: Acidaminococcaceae, Porphyromonadaceae, Lachnospiraceae, Ruminococcaceae, Bacteroidaceae, Eubacteriaceae, Erysipelotrichaceae, Coriobacteriaceae, Desulfovibrionaceae, Clostridiales Incertae Sedis XIII, Clostridiaceae Prevotellaceae, Verrucomicrobiaceae, Sutterellaceae, Defluviitaleaceae, Streptococcaceae, Bifidobacteriaceae, Clostridiaceae, and Leuconostocaceae.

Genus: Phascolarctobacterium, Barnesiella, Dorea, Blautia, Clostridium XlVa, Clostridium XlVb, Anaerorhabdus, Eubacterium, Bacteroides, Eggerthia, Collinsella, Flavonifractor, Ruminococcus, Gordonibacter, Bilophila, Anaerofustis, Mogibacterium, Coprococcus, Oscillibacter, Clostridium sensu stricto, Lachnospiraceae UCG-008, Lachnoclostridium, Eubacterium fissicatena group, Dielma, Marvinbryantia, Subdoligranulum, Lachnospiraceae NK3A20 group, Prevotella, Akkermansia, Odoribacter, Clostridium XVIII, Ruminococcus2, Parasutterella, Clostridium IV, Defluviitalea, Streptococcus, Bifidobacterium, Butyricicoccus, Clostridium sensu stricto, and Weissella.

Species: Phascolarctobacterium faecium, Blautia hansenii/producta, Clostridium XlVa bolteae/clostridioforme, Clostridium XlVa scindens, Bacteroides uniformis, Clostridium XlVa oroticum, Bacteroides ovatus, Bacteroides caccae, Bacteroides cellulosilyticus/timonensis, Bacteroides intestinalis, Collinsella aerofaciens, Blautia faecis, Anaerofustis stercorihominis, Clostridium sensu stricto celatum/disporicum, Lachnospiraceae UCG-008 uncultured organism, Bacteroides thetaiotaomicron, Marvinbryantia metagenome, Lachnospiraceae NK3A20 group uncultured bacterium, Akkermansia muciniphila, Bacteroides dorei/vulgatus, Bacteroides massiliensis, Coprococcus comes, Blautia obeum, Bacteroides faecichinchillae/faecis/thetaiotaomicron, Clostridium XlVa glycyrrhizinilyticum, Parasutterella excrementihominis, Streptococcus anginosus subsp. anginosus, Bacteroides uniformis, Bacteroides thetaiotaomicron, and Butyricicoccus uncultured bacterium.

Exemplary bacteria that increase in response to MTX:

Phyla: Firmicutes, Bacteroidetes, Proteobacteria, and Actinobacteria.

Class: Clostridia, Erysipelotrichia, Bacteroidia, Actinobacteria, Betaproteobacteria, NA, Deltaproteobacteria Bacilli, and Gammaproteobacteria Coriobacteriia.

Order: Clostridiales, Erysipelotrichales Bacteroidales, Coriobacteriales, Burkholderiales, NA, Desulfovibrionales, Lactobacillales, and Enterobacteriales.

Family: Lachnospiraceae, Peptostreptococcaceae NA, Ruminococcaceae, Erysipelotrichaceae, Bacteroidaceae, Coriobacteriaceae, Sutterellaceae, Rikenellaceae, Desulfovibrionaceae, Eubacteriaceae, Enterococcaceae, Tannerellaceae, Prevotellaceae, Porphyromonadaceae, Enterobacteriaceae, Streptococcaceae, Eggerthellaceae, Lactobacillaceae, Atopobiaceae, and Clostridiaceae 1.

Genus: Clostridium XlVa, Dorea, Ruminococcus, Clostridium XI, Blautia, Bacteroides, Enterorhabdus, Anaerosporobacter, Clostridium IV, Eggerthella, Ruminococcus, Faecalibacterium, Parasutterella, Butyricicoccus, Alistipes, Acetanaerobacterium, Bilophila, Oscillibacter, Eubacterium, Pseudoflavonifractor, Subdoligranulum, Enterococcus, Roseburia, Parabacteroides, uncultured, Paraprevotella, Lactonifactor, Escherichia/Shigella, Clostridium XVIII, Clostridium XlVb, Clostridium III, Escherichia-Shigella, Lactococcus, Gordonibacter, Senegalimassilia, Slackia, Lactobacillus, and Clostridium sensu stricto 1.

Species: Blautia obeum/wexlerae, Bacteroides stercoris, Dorea longicatena, Parasutterella excrementihominis, Bacteroides finegoldii, Alistipes finegoldii, Alistipes putredinis, Bilophila wadsworthia, Clostridium_XlVa symbiosum, Alistipes shahii, Pseudoflavonifractor capillosus, and Bacteroides ovatus.

As used herein, the phrase “comparing the abundance” refers to comparing the presence of and amount of a bacterial species or strain relative to a baseline or threshold amount or relative to a “before and after” scenario where the amount is measured before and after a specified treatment.

Methotrexate is used to treat severe psoriasis (a skin disease in which red, scaly patches form on some areas of the body) that cannot be controlled by other treatments. Methotrexate is also used along with rest, physical therapy, and sometimes other medications to treat severe active rheumatoid arthritis (RA; a condition in which the body attacks its own joints, causing pain, swelling, and loss of function) that cannot be controlled by certain other medications. Methotrexate is also used to treat certain types of cancer including cancers that begin in the tissues that form around a fertilized egg in the uterus, breast cancer, lung cancer, certain cancers of the head and neck, certain types of lymphoma, and leukemia (cancer that begins in the white blood cells). Methotrexate is in a class of medications called antimetabolites. Methotrexate treats cancer by slowing the growth of cancer cells. Methotrexate treats psoriasis by slowing the growth of skin cells to stop scales from forming. Methotrexate may treat rheumatoid arthritis by decreasing the activity of the immune system and, as described herein, by affecting the abundance certain species or strains of the gut microbiome.

Exemplary methotrexate medications include, but are not limited to, RASUVO®, TREXALL®, OTREXUP®, Methotrexate LPF Sodium, XATMEP®, JAMP-Methotrexate, METOJECT® and PMS-Methotrexate.

Methotrexate may be administered ion various dosages, according to the present disclosure including, but not limited to, 2.5 mg; 25 mg/mL; 25 mg/mL preservative-free; 1 g; 20 mg; 50 mg; 5 mg; 7.5 mg; 10 mg; 15 mg; 2.5 mg/mL; 10 mg/0.4 mL; 12.5 mg/0.4 mL; 15 mg/0.4 mL; 17.5 mg/0.4 mL; 20 mg/0.4 mL; 22.5 mg/0.4 mL; 25 mg/0.4 mL; 7.5 mg/0.4 mL; 7.5 mg/0.15 mL; 10 mg/0.2 mL; 12.5 mg/0.25 mL; 15 mg/0.3 mL; 17.5 mg/0.35 mL; 22.5 mg/0.45 mL; 25 mg/0.5 mL; 27.5 mg/0.55 mL; 30 mg/0.6 mL. One exemplary adult dose related to RA treatment provides: Single dose: 7.5 mg orally or subcutaneously once a week; Divided dose: 2.5 mg orally every 12 hours for 3 doses once a week; and Maximum weekly dose: 20 mg.

According to some embodiments of the present disclosure, it may be determined that methotrexate treatment will not aid the treatment of RA or other diseases described herein (i.e., the abundance of certain bacterial strains or species was not impacted by an initial trial of methotrexate treatment as described herein). Medications other than methotrexate that are contemplated by the present disclosure under such circumstances include, but are not limited to, nonsteroidal anti-inflammatory drugs (NSAIDs), steroids, disease-modifying antirheumatic drugs (DMARDs), and biological agents.

Exemplary NSAIDs include ibuprofen, naproxen sodium, diclofenac, meloxicam, sulindac, CELEBREX®, nabumeton, indomethacin, ketoprofen, and etodolac.

Exemplary steroids include, but are not limited to, prednisone, prednisolone, methylprednisolone, dexamethasone, hydrocortisone, triamcinolone, and betamethasone.

Exemplary DMARDs include, but are not limited to leflunomide (Arava), hydroxychloroquine (PLAQUENIL®), sulfasalazine (AZULFIDINE®), azathioprine (IMURAN®) and minocycline.

Exemplary biological agents include, but are not limited to, abatacept (ORENCIA®), adalimumab (HUMIRA®), anakinra (KINERET®), baricitinib (OLUMIANT®), certolizumab (CIMZIA®), etanercept (ENBREL®), golimumab (SIMPONI®), infliximab (REMICADE®), rituximab (RITUXAN®), sarilumab (KEVZARA®), tocilizumab (ACTEMRA®), tofacitinib (XELJANZ®), and upadacitinib (RINVOQ®).

As described herein, various embodiments of the disclosure provide methods for reducing inflammation in the gut and/or joints of a subject. In various embodiments, the subject is suffering from RA and/or inflammatory bowel disease (IBD). In other embodiments, the subject is suffering from spondyloarthritis, lupus, psoriasis, sarcoidosis, myositis, uveitis, and/or other autoimmune diseases.

In still other embodiments, a method of preventing or reducing the destruction of joints and/or organs in a subject is provided. In other embodiments, a method of improving a subject's responsiveness to MTX treatment is provided comprising the steps of administering a diet or medication that selectively reduces or inhibits the growth of one or more bacterial taxon in said subjects gut. In another embodiment, a method of determining the susceptibility to methotrexate (MTX) in a subject or a method of improving the response to MTX in a subject is provided, as described herein.

The present disclosure provides, in various embodiments, that perturbation of the human gut microbiome by a non-antibiotic drug reduces immune activation. Methotrexate, the first-line therapy for rheumatoid arthritis (RA) and a therapeutic agent to numerous other diseases and disorders, is sufficient to affect the growth of human gut bacteria in isolation, complex communities, and within the gastrointestinal tract. As described herein, MTX-induced shifts in the gut microbiota and thus can act, in part, through off-target effects on the gut microbiota. The post-MTX gut microbiome led to a reduction multiple cell types in both the mucosa and periphery, which included activated T cells, Th17, and IFN-γ+ T cells. These cell types are thought to play key roles in RA pathogenesis and are consistent with evidence that MTX decreases IL-17 levels. Remarkably, the observed drug-induced changes in microbial community structure were associated with patient response, providing a potential prognostic biomarker for accelerating the stable initiation of therapy and a first step towards determining which bacterial taxa contribute to or interfere with treatment outcomes.

The present disclosure including the below Examples demonstrate that the off-target effects of MTX on the human gut microbiota are far broader than previously appreciated Bolin, J. T., et al., (1982), J Biol Chem 257, 13650-13662; Maier et al., (2018), Nature 555, 623-628; Wood et al., (1961), Biochem Pharmacol 6, 113-124). While there was considerable variability between mouse experiments, common trends in the microbiota's response to MTX were observed across multiple donors, including healthy control and RA patients, and from the phylum to the ASV level. As described herein, microbiota shifts across oral and intraperitoneal dosing, consistent with prior reports of MTX enterohepatic circulation (Grim et al., (2003), Clin Pharmacokinet 42, 139-151; Steinberg, S. E., et al., (1982), Cancer Res 42, 1279-1282). Folic acid, which is commonly co-administered with MTX in RA, did not markedly rescue the gut microbiota, perhaps due to the absorption of this supplement in the proximal GI tract Visentin, M., et al., (2014), Annu Rev Physiol 76, 251-274). Similar studies in patient cohorts are needed to test the translational relevance of these findings and to evaluate additional factors that could shape the sensitivity of the gut microbiota to MTX and other immunomodulatory drugs, including inter-individual variations in drug disposition (Hoekstra, M., et al., (2004), J Rheumatol 31, 645-648), genetic risk factors (Weyand, C. M., et al., (1995), J Clin Invest 95, 2120-2126), and baseline microbial community structure or function Nat Med 21, 895-905).

While the effects of MTX were detectable across multiple bacterial phyla, just a single phylum (the Bacteroidetes) was consistently affected in mice, bacterial isolates, mixed communities, and non-responsive patients. The underlying determinants of variability in MTX sensitivity at the cellular, community, and ecosystem level remain to be investigated, are likely multifactorial, and may involve one or more of the following: drug influx and efflux Kopytek, S. J., et al., (2000), Antimicrob Agents Chemother 44, 3210-3212), drug metabolism (Valerino, D. M., et al., (1972), Biochem Pharmacol 21, 821-831), compensatory pathways (i.e., de novo synthesis of folic acid) (Wood, R. C., et al., (1961), Biochem Pharmacol 6, 113-124), and redundancies in the cellular pathways to produce reduced folates (Myllykallio, H., et al., (2003), Trends Microbiol 11, 220-223), as well as microbe-microbe or host-microbe interaction. Consistent with the importance of these higher-order interactions, ASVs were detected with discrepant sensitivities in vitro and in vivo (e.g., Alistipes shahii). From a translational perspective, the observation of reproducible findings at the phylum level may be a simple and useful biomarker of therapeutic efficacy, which would complement ongoing attempts to integrate data on the pre-treatment microbiome with more established risk factors like host genetics and smoking status (Halilova, K. I., et al., (2012), Int J Rheumatol 2012, 978396).

The in-depth analysis of select Firmicutes and Bacteroidetes provided herein isolates is consistent with the hypothesis that MTX acts by inhibiting bacterial DHFR with broad downstream consequences for purine and pyrimidine biosynthesis among other cellular pathways, including amino acid biosynthesis and replication. The results described herein are consistent with data from human cells (Allegra, C. J., et al., (1987), J Biol Chem 262, 13520-13526; Genestier, L., et al., (2000), Immunopharmacology 47, 247-257), wherein DHFR inhibition leads to changes in the expression of purine and pyrimidine pathways, which rely on folate as a co-factor for multiple key reactions. Rescue experiments with folic acid and leucovorin further support this observation; however, the existence of partial and opposite effects underscore both the broader effects of MTX beyond targeting of bacterial DHFR and the complexities of interactions between metabolites (e.g., folate) and anti-metabolite drugs (e.g., MTX). Even in the absence of growth inhibition, there were marked changes in transcriptional and metabolic activity, consistent with our prior work on other non-antibiotic drugs Maurice, C. F., et al., (2013), Cell 152, 39-50). These results emphasize the importance of considering the broader impacts of drugs on gut microbial physiology and metabolic activity, even in the absence of marked changes in community structure or colonization level.

As disclosed herein, microbiome transplantations into germ-free mice given an inflammatory stimulus provided evidence that MTX-induced changes in the gut microbiota lead to changes in immune cell levels. This includes a reduction in multiple cell populations in the periphery, including activated T cells, IFN-γ+ T cells, myeloid cells, and B cells. Reductions in activated T cells, Th17 cells and myeloid cells in the intestinal mucosa were also found. Th1, Th17 and B cells have been previously implicated in RA pathogenesis (Imboden, J. B. (2009), Annu Rev Pathol 4, 417-434) and prior studies suggest that MTX decreases IL-17 levels in RA patients (Yue, C., et al., (2010), Rheumatol Int 30, 1553-1557), consistent with our observed decreases in Th17 cells. Reduced ileal Foxp3+in an unchallenged state was observed, suggesting a tonic reduction of Tregs (a T cell population that reduces inflammation) perhaps as a result of reduced post-MTX microbiota mediated stimulation of the immune system and the reduced need for tolerogenic induction of Tregs (Omenetti, S., and Pizarro, T. T. (2015), Front Immunol 6, 639). Taken together, with the majority of changes (10/12 or 83%) exhibiting a reduction in post-MTX recipient mice, these findings suggest that MTX-induced shifts to the microbiota reduce its inflammatory potential. Additional studies are needed in gnotobiotic mouse models of RA (Maeda, Y., et al., (2016), Arthritis Rheumatol 68, 2646-2661) coupled to genetic and pharmacological immune perturbations to dissect the host pathways responsible.

The computational analyses provided herein have identified multiple candidate bacterial species that are consistent with the prior literature. This includes the model Bacteroidetes B. theta (ASV23), which is sensitive to MTX, positively associated with immune activation, and exacerbates a mouse model of colitis (Hickey, C. A., et al. (2015), Cell Host Microbe 17, 672-680). Other Bacteroidetes may also be important, given their ability to activate Thl cells in the context of intestinal parasitic infection (Heimesaat, M. M., et al. (2006), J Immunol 177, 8785-8795). Of note, Prevotella copri (ASV914), which is a Bacteroidetes member and has been previously associated with RA and Th17 activation (Maeda, Y., et al., (2016), Arthritis Rheumatol 68, 2646-2661; Pianta, A., et al., (2017), Arthritis Rheumatol 69, 964-975), was decreased with MTX treatment in gnotobiotic mice and ex vivo communities, as well as post-MTX recipient mice. Another promising candidate that has previously been associated with RA is the gut Actinobacterium Collinsella aerofaciens (ASV44), which was sensitive to MTX in multiple transplant experiments and aggravates inflammatory arthritis via Th17 (Chen, J., et al., (2016), Genome Med 8, 43). Other candidates include Dielma fastidiosa (ASV72), ASV908 (Paraprevotella) and ASV296 (Lachnoclostridium) which were directly impacted by MTX and showed associations with immune cell phenotypes.

The present disclosure thus emphasizes the importance of taking a broader view of pharmacology that encompasses the unintended consequences of non-antibiotic drugs for our associated microbial communities. The present disclosure demonstrates the utility of integrated studies in vitro, in gnotobiotic mice, ex vivo, and in drug naïve patients to begin to elucidate the causality and mechanism for these complex drug-microbiome-host interactions. MTX-induced changes in microbial community structure were associated with patient response, providing a biomarker for accelerating the stable initiation of therapy and a first step towards determining which bacterial taxa contribute to or interfere with treatment outcomes.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

The following materials and methods were used in in the Examples described herein.

### Gnotobiotic Mouse Studies

C57BL/6J mice (females, ages 6-16 weeks) were obtained from the UCSF Gnotobiotics core facility (gnotobiotics.ucsf.edu) and housed in gnotobiotic isolators for the duration of each experiment (Class Biologically Clean). Key details for each gnotobiotic experiment can be found in Table S1B. Housing: Mice were co-housed in some experiments, distributed across multiple cages per treatment group, or individually housed. Individually housed mice were housed in Single Mouse Duplexed Cages (Thoren, Cat #15). Colonization: Mice were colonized with stool from human donors, either a healthy male donor or a treatment-naive (re-MTX) female and male donors with rheumatoid arthritis [as defined by American College of Rheumatology classification criteria, either (Arnett, F. C., et al. (1988), Arthritis Rheum 31, 315-324) or (Aletaha, D., et al. (2010), Arthritis Rheum 62, 2569-2581)]; samples obtained 1 month after starting MTX were used for “post-MTX” transplants. For colonization with a human microbiome, stool was diluted 1:10 g/mL in reduced PBS or saline and homogenized in an anaerobic chamber using pre-equilibrated reagents and supplies. Insoluble material was separated from supernatant by centrifugation at 50 g for 1 minute. Aliquots of supernatant (100-200 μl per mouse) were gavaged into mice. In fecal transplant experiments using pre-MTX and post-MTX samples from RA donors, MTX at the limit of quantification (100 nM) was not detected using targeted LC-MS, particularly in the post-MTX samples, suggesting that only microbiota (and not drug) was being transferred into recipient mice. Mice were colonized for at least 1-2 weeks before initiation of treatment with MTX (Pharmaceutical-grade, Fresenius Kabi, NDC 63323-122-50, Product #102250). Mice were colonized for at least 2 weeks prior to dextran sodium sulfate treatment (DSS) treatment. Stool samples were collected on days 3, 7-8, and 14 prior to DSS treatment. Drug treatment: In experiments examining MTX treatment, mice were treated either with saline/PBS, MTX 1 mg/kg or 50 mg/kg daily or folic acid 50 mg/kg. Treatment was carried out either by oral gavage or intra-peritoneal injection. Mice were monitored and weighed daily during treatment. No gross signs of toxicity and minimal weight loss were observed for the short MTX treatment durations used in this study. Stool samples were collected daily during treatment. Duration of treatment with MTX was 4 days in the dose-response experiment and in individually housed mice. For the route and rescue experiments, duration of treatment was reduced to 2 days because in the dose-response experiment, with high-dose MTX, changes in community composition were seen within 1-2 days so subsequent treatments with high-dose MTX were done for 2 days given the large effect size seen with a single day of treatment. According to the UCSF Animal Studies protocols, adherence to the three R's of animal research were followed (Guhad, F. (2005), Contemp Top Lab Anim Sci 44, 58-59), and in this case, the “refinement” principle was implemented, which advocates that researchers modify “husbandry or experimental procedures to minimize pain and distress, and to enhance the welfare of an animal used in science from the time it is born until its death.” Upon completion of treatment, mice were euthanized, and samples from the following tissues were harvested: 18 segment of the ileum and its contents (unconcentrated), cecal tissue and contents, and distal colon tissue and its contents. In DSS experiments, colon length was measured prior to sectioning of the colon. For DSS treatment (Alfa Aesar, Cat no. 9011-18-1), mice were given 2% DSS (w/v) ad libitum in their drinking water for 6-8 days, and DSS water was replaced on days 3 or 4. DSS was used as an inflammatory trigger to challenge the immune system instead of a murine arthritis model because of several favorable properties of the DSS model combined with the inherent difficulties of murine arthritis models in gnotobiotic mice: (1) DSS is highly penetrant in gnotobiotic mice, (2) DSS has been used to understand host-microbiota effects in inflammatory arthritis (Scher, J. U., et al. (2013), Elife 2, e01202), (3) DSS has a rapid onset, and (4) many murine models of arthritis rely on a genetic abnormality which would need to be rederived in a gnotobiotic setting (Vincent, T. L., et al., (2012), Rheumatology (Oxford) 51, 1931-1941). All mouse experiments were approved by the University of California San Francisco Institutional Animal Care and Use Committee.

### NYU Human RA Patient Samples Acquisition

Consecutive patients from the New York University Langone Medical Center's rheumatology clinics and offices were screened for the presence of RA based on ACR criteria (Aletaha et al., 2010). After informed consent was signed, each patient's medical history (according to chart review and interview/questionnaire), diet, and medications were determined. A screening musculoskeletal examination and laboratory assessments were also performed or reviewed. All RA patients who met the study criteria were offered enrollment. The criteria for inclusion in the study required that patients meet the American College of Rheumatology/European League Against Rheumatism 2010 classification criteria for RA (Aletaha et al., 2010), including seropositivity for rheumatoid factor and/or anti-citrullinated protein antibodies, and that all subjects be age 18 years or older. New-onset RA was defined as disease duration of a minimum of 6 weeks and up to 6 months since diagnosis, and absence of any treatment with disease-modifying anti-rheumatic drugs, biologic therapy or steroids (ever). The exclusion criteria applied to all groups were as follows: recent (<3 months prior) use of any antibiotic therapy, current extreme diet (e.g., parenteral nutrition or macrobiotic diet), known inflammatory bowel disease, known history of malignancy, current consumption of probiotics, any gastrointestinal tract surgery leaving permanent residua (e.g., gastrectomy, bariatric surgery, colectomy), or significant liver, renal, or peptic ulcer disease. This study was approved by the Institutional Review Board of New York University School of Medicine protocols #09-0658 and as previously published (Scher et al., 2013). All new onset rheumatoid arthritis (NORA) patients were recruited using established protocols from a previously described study (Scher et al., 2013). Patients received oral MTX at standard of care doses as prescribed by their treating rheumatologists. Stool samples were collected at baseline and 1 month after MTX initiation and metadata were obtained at baseline and 4 months after therapy initiation. Clinical and demographic data was de-identified and recorded in RedCap by the designated study personnel. Clinical responder status (MTX-R) was defined a priori as any NORA patient whose DAS28 score was greater than 2 at baseline and improved by at least 1.8 by month 4 post-treatment. DNA was extracted from human fecal samples (n=23 patients, 46 stool samples) as previously described (Scher et al., 2013), using the MoBio Powersoil DNA extraction kit, based on cell membrane disruption by high-speed shaking in the presence of beads. The V4 hypervariable region of bacterial 16S ribosomal RNA (rRNA) was performed using a MiSeq 11lumina platform (150-bp read length, paired-end protocol) at the New York University Genome Technology Center as previously described (Manasson et al., 2018). For each sample, fastq files are available in NCBI's Sequence Read Archive (SRA), accession number PRJNA656577.

### MTX Dose Selection in Animal Models

The doses used in this study were selected based upon a careful consideration of the doses used previously in humans and mice. While it is dangerous to overinterpret the relevant doses in mice to any human disease, the current gold standard is based upon allometric scaling ( Nair, A. B., and Jacob, S. (2016), J Basic Clin Pharm 7, 27-31). Based on this simple conversion (animal dose×0.081=human dose), our two doses are the equivalent to 81 μg/kg and 4.05 mg/kg in humans. Given the average 60 kg humans, this equates to 4.86 mg/day and 243 mg/day, respectively. According to Mayo Clinic (www.mayoclinic.org/drugs-supplements/methotrexate-oral-route/proper-use/drg-20084837), doses in for cancer patients range from 5-50 mg/day, while doses for rheumatoid arthritis are typically ˜7.5-20 mg/day (administered once per week). Thus, the low dose is slightly lower than the lower end of the human dosing regimens, whereas our high dose is higher than what is typically used in patients. However, the doses were not overinterpreted used herein, given the potential differences in MTX metabolism, absorption, clearance, and distribution between humans and mice that could complicate these matters, in addition to microbiome-dependent factors such as the differences in the ability of bacteria to convert MTX to DAMPA (Valerino, D. M., et al., (1972), Biochem Pharmacol 21, 821-831) and/or other metabolites. Additional studies examining pharmacokinetics in mice and patients are needed to capture the genetic and microbiologic factors that could influence drug disposition. Thus, quantitative comparisons of dose equivalency in humans and mice remain highly speculative at this time.

### 16S-seq of Humanized Mouse Gut Microbiota

Broadly, aliquots of mouse fecal, ileal, cecal and colon samples (Table S2A) were homogenized using a bead-beating (Mini-Beadbeater-24, BioSpec) method followed by DNA extraction and purification. For the dose-response gnotobiotic experiment, bead beating was achieved using Lysing Matrix E 2 mL Tube beds (MP Biomedicals) and using the digestion solution and lysis buffer of a Wizard SV 96 Genome DNA kit (Promega). The samples were then centrifuged for 10 minutes at 16,000 g and the supernatant was transferred to the binding plate. The DNA was purified according to the manufacturer's instructions. DNA from the remaining experiments was extracted using ZymoBIOMICS 96 well MagBead ZymoBIOMICS 96 MagBead DNA Kit (Cat #D4302) as per the manufacturer's protocol.

GoLay-barcoded 515F/806R primers (Caporaso, J. G., et al. (2012), ISME J 6, 1621-1624) were used to carry out 16S rRNA gene PCR according to the methods of the Earth Microbiome Project (earthmicrobiome.org). For the dose-response experiment, the following were combined: 2 μL of DNA, 25 μL of AmpliTaq Gold 360 Master Mix (Life Technologies), 5 μL of primers (2 μM each GoLay-barcoded 515/806R), and 18 μL H2O. Amplification was as follows: 10 minutes at 95° C., 25×(30 seconds at 95° C., 30 seconds at 50° C., 30 seconds at 72° C.), and 7 minutes at 72° C. For the dose-response gnotobiotic experiment, amplicons were quantified with PicoGreen (Quant-It dsDNA; Life Technologies) and pooled at equimolar concentrations. Libraries were quantified (NEBNext Library Quantification Kit; New England Biolabs) and sequenced with a 600 cycle MiSeq Reagent Kit (251×151; Illumina) with ˜10% PhiX. For the remaining sequencing experiments, samples underwent primary PCR for amplification and secondary PCR to add flow cell adaptors and indices as previously described (Gohl, D. M., et al. (2016), Nat Biotechnol 34, 942-949). Normalization was achieved using SequalPrep Normalization (Life Tech A10510-0) kits. Pooled libraries were purified and concentrated with MinElute PCR Purification kit (Qiagen #28004), run on 1% gel, size-selected and purified using MinElute Gel Extraction kits (Qiagen, #28604). Pooled libraries were run at the Chan Zuckerberg Biohub using Illumina MiSeq platform.

### In Vitro Bacterial Growth Studies

42/45 of the tested isolates are commonly found in the human gut microbiota, with the exceptions of Bacteroides acidifaciens (found in mice), Delftia acidovorans (found in soil), and Bacillus subtilis 168 (found in soil). Each of these strains was obtained from the Deutsche Sammlung von Mikroorganismen and Zellkulturen (DSMZ) culture collection. A single colony of each isolate was subcultured in Bacto Brain Heart Infusion (BD Biosciences, 37 g/L) supplemented with L-cysteine-HCl (0.05%, w/v), menadione (1 μg/mL), and hemin (5 μg/mL) (referred to hereafter as BHI+) for 48 hours in an anaerobic chamber (Coy Laboratory Products) at 37° C. with an atmosphere composed of 2-3% H₂, 20% CO₂, and the balance N₂. This subculture was diluted down to an OD600 of 0.08-0.1, which was then further diluted 100-fold, and then used to inoculate a microtiter plate with 2-fold serial dilutions of MTX concentrations ranging from 0-900 μg/ml. Plates were incubated at 37° C. with shaking in an Eon Microplate Spectrophotometer (BioTek Instruments, Inc) over a 48 to 72-hour period in the anaerobic chamber. Growth was monitored every 15 minutes at OD600 and corrected for background (no growth control). Data were exported using the Gen5 (v 2.0) software. The minimal inhibitory concentration (MIC) was measured as the lowest concentration of MTX resulting in >90% growth inhibition after 48 hours of incubation. Growth curves were not available for 6 of the studied isolates due to technical issues but were still able to assess MIC at 48 hours based on visual inspection and endpoint OD600 measurement. As with any in vitro system, the growth of cells may be dependent on the growth medium used, and for these studies, a rich medium (BHI+) was used.

In Vitro Bacterial Rescue Studies with Folic Acid and Leucovorin

As described in the ur in vitro bacterial growth studies, 48-hour overnight cultures were grown in an anaerobic chamber in liquid BHI+ for the following isolates: Bacteroides vulgatus, Bacteroides thetaiotaomicron, Clostridium innocuum and Clostridium symbiosum. Each subculture was diluted down to an OD600 of 0.08-0.1, which was then further diluted 100-fold, and then used to inoculate a 96-well microtiter plate with 2-fold serial dilutions of MTX (Sigma, Cat #M9929) concentrations ranging from 0-450 μg/ml (0-990 μM) along the columns (1-10) and 2-fold serial dilutions of folic acid (Fischer Scientific, BP25195) ranging from 0-250 μg/ml (0-566 μM) along the rows (A-H). Each plate included media and growth controls. The same setup was used to test the rescue effects of 2-fold serial dilutions of leucovorin (Spectrum Chemicals, Cat #LE117) at concentrations ranging from 0-125 μg/ml (0-264 μM) and the following isolates were tested: B. vulgatus, B. thetaiotaomicron, C. innocuum, and C. symbiosum. Solubility limits restricted the max concentrations for each compound.

### Tree Construction

Full-length ribosomal sequences for each isolate were extracted from the Greengenes (DeSantis, T. Z., et al., (2006), Appl Environ Microbiol 72, 5069-5072) database (May, 2013). Sequences were imported into UGENE (Okonechnikov et al., 2012) (v 1.31.0), and aligned using MUSCLE (Edgar, 2004). Gaps occurring in >50% of sequences were removed, and a maximum likelihood tree was generated using PhyML (Guindon, S., et al., (2010), Syst Biol 59, 307-321) with 100 bootstraps and the GTR substitution model. For trees generated from 16S-seq from gnotobiotic mice or ex vivo RA samples, the ggtree R package was used(v 2.0.2)(Yu, G., et al., (2018), Mol Biol Evol 35, 3041-3043).

### Estimated Abundance of Bacterial Species in Human Metagenomes

Bacterial abundances were quantified using data from MetaQuery (Nayfach, S., et al., (2015), Bioinformatics 31, 3368-3370), a web-based application that provides taxonomic abundances from >1,900 publicly available human gut metagenomes. For each isolate, the “metaphlan2” database was queried and recorded the mean abundance value. The version of MetaPhlAn2 used by MetaQuery was 2.2.

### Predicted Concentration of MTX in the Gastrointestinal (GI) Tract

The predicted concentration of MTX in the proximal GI tract was estimated by taking the oral dose used for rheumatoid arthritis (25 mg) and dividing it by 250 ml Dahlgren, D., and Lennernas, H. (2019), Pharmaceutics 11, 411; Dressman, J. B., et al., (1985), J Pharm Sci 74, 588-589; Shekhawat, P.B., and Pokharkar, V. B. (2017), Acta Pharm Sin B 7, 260-280), giving a concentration of 100 μg/ml or 220 μM. Further refinement of this estimate was done based on variations in the absorption of oral MTX in the proximal intestine (Grim, J., et al., (2003), Clin Pharmacokinet 42, 139-151), where it is estimated that 10-70% of the dose is not absorbed and instead delivered to the distal GI tract in RA patients. Thus, the estimated range of MTX in the GI tract was calculated to be 10-70 μg/ml with 100 μg/ml being the highest predicted concentration (prior to absorption by the host). When considering the doses used for cancer therapy (50 mg/day of oral MTX), the predicted range is 20-140 μg/ml with 200 μg/ml being the highest dose. MTX can also be given intravenously (IV) or intrathecally for the treatment of cancer (Treon, S. P., and Chabner, B. A. (1996), Clin Chem 42, 1322-1329), and is typically given at much higher doses, including as high as 800-1000 mg IV over 24 hours for the treatment of cancers such as lymphoma or acute lymphoblastic leukemia. Assuming that 30% of this is enterohepatically circulated et al., (1982), Cancer Res 42, 1279-1282), then between 240 and 300 mg would enter the GI tract, and the predicted concentration in the GI tract would be between 960-1200 μg/ml.

### MTX Treatment for RNA-seq

The bacterial strains used in RNA-seq are given in Table S4. Genomes were obtained from NCBI's GenBank Assembly database. Culture media was composed of BHI+and allowed to equilibrate in an anaerobic environment prior to use. Briefly, bacteria were cultured in BHI+at 37° C. in an anaerobic chamber. Cultures for each isolate were grown to mid-exponential (achieving an OD600˜0.5), aliquoted into triplicates, treated for 30 minutes with either DMSO or MTX 100 μg/ml, and then removed from the anaerobic chamber. The mid-exponential time point was selected to facilitate comparisons across isolates, as has been done by others (O'Rourke, A., et al. (2020), Antimicrob Agents Chemother 64), since lag and stationary phases would present challenges in term of biomass (lag phase with low biomass) and differing physiology (i.e., sporulation among some isolates and not others). For C. asparagiforme, cultures were incubated for 4 and 20 hours as well and profiled. Cultures were centrifuged at 2000 rpm for 10 minutes at 4° C. to facilitate removal of supernatant, and the remaining bacterial pellet was flash-frozen in liquid nitrogen.

### Total RNA Extraction

Each bacterial pellet was incubated with 1 ml of Tri reagent (Sigma Aldrich, catalog #: T9424) at room temperature for 10 minutes. The cell suspension was transferred into Lysing Matrix E tubes (MP Biomedicals, 116914050), and homogenized in a bead-beater (Mini-Beadbeater-24, BioSpec) for 5 minutes at room temperature. The sample was incubated with 200 μL of chloroform at room temperature for 10 minutes, followed by centrifugation at 16,000×g for 15 minutes at 4° C. Next, 500 μL of the upper aqueous phase was transferred into a new tube and 500 μL of 100% ethanol was added. To isolate RNA, PureLink RNA Mini Kit (Life Technologies, catalog #: 12183025) was used. This mixture was transferred onto a PureLink spin column and spun at >12,000×g for 30 seconds. The column was washed with 350 μl of wash buffer I as described in the PureLink manual. The column was incubated with 80 μl of PureLink DNase (Life Technologies, catalog #: 12185010) at room temperature for 15 minutes, and washed with 350 μl of wash buffer I. The column was washed with wash buffer II twice as described in the PureLink manual. Total RNA was recovered in 50 μl of RNAase-free water. A second round of off-column DNAse treatment was undertaken. The RNA was incubated with 6 μl of TURBO DNAse (Ambion, ThermoFisher, catalog #: AM2238) at 37° C. for 30 minutes. To stop the reaction, 56 μl of lysis buffer from the PureLink kit and 56 μl of 100% ethanol was added to the sample and vortexed. This suspension was transferred onto a PureLink column and washed once with 350 μl of wash buffer I and twice with 500 μl of wash buffer II. The RNA was recovered in 30 μl of RNAse-free water.

rRNA Depletion, Library Generation, and RNA Sequencing

Total RNA was subjected to rRNA depletion using Ribo-Zero Bacterial rRNA Depletion (Illumina, catalog #: MRZB12424), following the manufacturer's protocol. RNA fragmentation, cDNA synthesis, and library preparation proceeded using NEBNext Ultra RNA Library Prep Kit for Illumina (New England BioLabs, catalog #: E7530) and NEBNext Multiplex Oligos for Illumina, Dual Index Primers (New England BioLabs, catalog #: E7600), following the manufacturer's protocol. All samples were single end sequenced (1×50 bp) using an Illumina HiSeq2500 platform (High Output, v4 chemistry) at UCSF's Institute for Human Genomics. For each sample, fastq files are available in NCBI's Sequence Read Archive (SRA), accession number PRJNA656577.

### Untargeted Metabolomics of In Vitro Cultures

As described in the in vitro bacterial growth studies, 48-hour overnight cultures were grown in an anaerobic chamber in liquid BHI+for the following isolates: Bacteroidetes thetaiotaomicron and Clostridium asparagiforme. Each subculture was diluted down to an OD600 of 0.08-0.1, which was then further diluted 100-fold, and then used to inoculate a 96-well 2-ml deep well plate with 100 μg/ml MTX (n=6 replicates per isolate) (Sigma, Cat #M9929) or DMSO (n=2 replicates per isolate). After 24 hours, aliquots were removed, centrifuged on a tabletop centrifuge at 4° C. for 5 minutes at ˜2000 rcf, and supernatant was prepared for extraction and untargeted LC-MS as described.

### Untargeted Metabolomics Sample Processing

Extraction was done in 96 well plates. In brief, 60 μL of the supernatant from the 96 well culture plate was added to 120 μL of pre-cooled methanol/acetonitrile (1:1, v/v) in a non-sterile 96 well plate. The samples were then incubated at −20° C. for 1 hour. After incubation, samples were mixed at 4° C. at 650 rpm using small block tube shaker. The plates were then centrifuged at 3000 rpm for 10 minutes. After centrifugation, using multi-channel pipette, 80 μL of supernatant from each well was transferred to non-sterile 96 well polypropylene plate and then sealed with pre-slit silicone mat. A 1:1:1 mix of LC-MS grade H₂O, LC-MS grade acetonitrile and LC-MS grade methanol was used as blank.

### UPLC-MS Experiments for Untargeted Metabolomics Data Acquisition

Samples (5 μl) were separated by hydrophilic interaction chromatography HPLC using a Vanquish UHPLC system (Thermo Fisher Scientific, Waltham, MA) with a Waters (Milford, MA) Acquity UPLC BEH Amide Column (2.1×100 mm×1.7 μm particle size) maintained at 65° C. and a 17 minute gradient, at a flow rate of 400 μl/min. Solvent A was 100% HPLC grade water with 0.1% formic acid and 10 mM ammonium formate, Solvent B was 95% HPLC grade acetonitrile and 5% HPLC grade water with 0.1% formic acid and 10 mM ammonium formate. The initial condition was 100% B, decreasing to 70% B at 7.7 minutes, 40% B at 9.5 minutes, 30% B at 10.25 minutes, and 100% B at 12.750 minutes where it was held until 17 minutes. The eluate was delivered into an Orbitrap Fusion Lumos TribridTM mass spectrometer using a H-ESI™ ion source (all Thermo Fisher Scientific). The mass spectrometer was scanned from 75-750 m/z at a resolution of 120,000 and operated in polarity switching mode. The capillary voltage was set at 3.25 kV in positive ion mode, and 2.025 kV in negative ion mode with an RF lens value of 30%, and an AGC target of 1.0E+6 with a maximum injection time of 50 ms. Product ion MS/MS spectra were acquired in AcquireX mode, with one exclusion blank, then three subsequent sample injections. The parent ion was isolated using the quadrupole with an isolation window of 1.5 Da, a stepped HCD (10,25,40 V) was used for activation, the mass spectrometer was operated at a resolution of 7500, and 3 microscans were acquired for each MS2 spectra.

### Metabolomics Dataset Processing

The raw data were converted into mzML format using ProteoWizard v3.0. Converted HILIC files were then analyzed using MS-DIAL v4.16 for deconvolution, peak detection, alignment, and identification (Parameters file can be provided upon request). MassBank of North America (MoNA) reference database was used for identification. Alignment results were exported and then normalized by TIC of each sample. Statistical analyses were done using R (v4.0.1) as described below.

### Ex Vivo Incubation of RA Patient Tool Samples

All work was carried out in an anaerobic chamber. For each patient, stool was aliquoted into a pre-equilibrated cryovial, diluted in reduced PBS at 10 ml per 1 gram of stool, and vortexed to homogenize the sample. The sample was spun at ˜20 g for 1 minute on a mini-centrifuge to facilitate settling of sediment, and the sediment-free supernatant was then aliquoted into a new pre-equilibrated cryovial for evaluation of ex vivo growth. Growth was evaluated by inoculating liquid BHI with 1:50 dilution of this fecal slurry, with OD600 readings performed every 15 minutes for 48 hours with a 2-minute shake prior to each reading. Samples were treated with MTX 100 μg/ml or an equal volume of DMSO at time zero. Each patient's fecal slurry and treatment was evaluated in quadruplicate. Samples from four individuals (Donors 2, 4, 5, and 6) underwent 16S sequencing and analysis at 0 and 24 hours after of treatment with either DMSO or MTX treatments (4 replicates per treatment group) as described in the section titled “16S-seq of humanized mouse gut microbiota.”

### Shotgun Sequencing of Rheumatoid Arthritis Patient Samples

Stool samples from 17 RA patients underwent DNA extraction using ZymoBIOMICS 96 MagBead DNA Kit (Cat #D4302) as per the manufacturer's protocol. Sequencing libraries were generated using the Nextera DNA Flex Library Kit (Illumina, #20018705) and Nextera Compatible Unique Dual Indices—Set A primers (Illumina, #20027213). Library concentrations were normalized using PicoGreen (Quant-It dsDNA; Life Technologies) and pooled for sequencing on the NovaSeq using S1 or S2 platform at the Chan Zuckerberg Biohub.

### Colon Histology

Approximately 1 cm sections from the distal colon were collected for histology from mice treated with or without DSS in the fecal microbiota transplant (FMT) studies. Samples were fixed in formalin for at least 24 hours and subsequently stored in 70% ethanol. Samples were processed by the UCSF Biorepository and Tissue Biomarker Technology Core. Tissues were embedded in wax and 4 μm cross-sections were H & E stained.

### Lamina Propria Lymphocyte Isolation

Lamina propria lymphocytes were isolated with slight modifications of previously described methods (Atarashi et al., 2011; Kubinak et al., 2015; Round et al., 2011). In brief, small intestinal (SI) Peyer's patches were removed and colons and SI tissue were splayed longitudinally with mucus removed by scraping and stored in complete RPMI (10% fetal bovine serum, 100 units per ml penicillin and streptomycin, β-mercaptoethanol, glutamate, sodium pyruvate, HEPES and non-essential amino acids). Supernatants were removed by filtering through a 100 μM filter, and remaining tissue incubated in 1× HBSS (without Ca²⁺ and Mg²⁺) containing 5 mM EDTA (Promega) and 1 mM DL-Dithiothreitol (DTT) (Bioplus chemicals) for 45 minutes at 37° C. on a shaker. Supernatant was removed by filtering through a 100 μM filter, and remaining tissue was incubated for 45 minutes (colon) or 35 minutes (SI) at 37° C. on a shaker in a solution containing 1× HBSS containing 5% (v/v) fetal bovine serum (GIBCO heat inactivated), 1 U/ml Dispase (Sigma), 0.5 mg/ml Collagenase VIII (Sigma), and 20 m/ml DNasel (Sigma). The supernatant was filtered over a 40 mm cell strainer into ice-cold sterile 1× PBS. Cells were subjected to a Percoll (VWR) gradient (40%/80% [v/v] gradient) and spun at 2000 rpm for 20 minutes with no brake and no acceleration. Cells at the interface were collected, washed in PBS and prepared for flow cytometry analysis.

### Flow Cytometry

Since RA pathophysiology is associated with a dysregulated T cell response (Imboden, J. B. (2009), Annu Rev Pathol 4, 417-434.), the T cell compartment in the spleen and the intestinal lamina propria of the small intestine and colon using flow cytometry were considered. Lymphocytes were isolated from the colonic and small intestinal lamina propria as described above. Spleen cells were prepped through gentle mashing with a syringe plunger. Spleen cells were treated with 1× RBC Lysis Buffer (Biolegend) to lyse and remove red blood cells. Surface staining for lymphocytes was done in staining buffer (1× HBSS (Corning) supplemented with 10 mM HEPES (Cellgro), 2 mM EDTA (Promega), and 0.5% (v/v) fetal bovine serum (GIBCO heat inactivated) for 20 minutes at 4° C. Cells were then washed twice in supplemented 1× HBSS and enumerated via flow cytometry. The following antibodies were used: anti-CD3 (17A2, Invitrogen, Cat. 11-0032-82), anti-CD4 (GK1.5, Biolegend, Cat. 100428), anti-CD69 (H1.2F3, Biolegend, Cat. 104511-BL), anti-CD11b (M1/70, Biolegend, Cat. 101228-BL), anti-CD44 (IM7, Tonbo biosciences, Cat. 10050-486), anti-Grl (clone RB6-8C5, Life Technologies, Cat. 108408), anti-TER119 (APC clone TER-119, Biolegend, Cat. 116212), and anti-B220 (clone RA3-6B2, Invitrogen, Cat. 5014055). For intracellular staining, cells were first stimulated with ionomycin (1000 ng/ml), PMA (50 ng/ml), and Golgi Plug (1 μl/sample) (BD Bioscience) overnight at 37° C. Stimulated cells were stained with LIVE/DEAD Fixable Aqua Dead Cell Stain Kit (Thermo Fisher) CD3+CD4+ cells were assessed within the live population. Cells were surface stained, washed, and then fixed/permeabilized in 100 μl Perm/Fix buffer (BD Bioscience). Cells were washed twice in Perm/Wash buffer (BD Bioscience) and then stained for intracellular cytokines with the following antibodies: anti-IFN-γ (XMG1.2, Millipore, Cat. MABF151), anti-IL-17A (ebio17B7, Invitrogen, Cat. 25-7177-82), anti-Foxp3 (150D, Biolegend, Cat. BDB563902). Cells were washed twice in Perm/Wash buffer and then placed in staining buffer for flow cytometry analysis. Gating cell populations was done using isotype and single stain controls.

### Quantification and Statistical Analysis

### General

No statistical methods were used to pre-determine sample sizes. All statistical analyses were performed using the R environment. “N.S.” or “ns” indicates “not significant.”

16S rRNA Amplicon Analysis of Mouse and Human Samples

Reads that were not already demultiplexed were demultiplexed using QIIME (Caporaso et al., 2010) v1.9.1 (split_libraries_fastq.py). For all reads except NYU RA patient samples, QIIME2 (Bolyen et al., 2019)(v 2020.2) was used to trim reads, denoise the data, and create a feature table using the following commands: qiime cutadapt trim-paired, qiime dada2 denoise-paired, qiime feature-table filter-samples. Within QIIME2, taxonomy was assigned using the DADA2 et al., (2016), Nat Methods 13, 581-583) implementation of the RDP Naïve Bayesian classifier (Wang, Q., et al., (2007), Appl Environ Microbiol 73, 5261-5267) using the DADA2-formatted SILVA v128 training set; taxonomic assignments to the species level were chosen by exact match of the ASV to the reference database, and where multiple matches were present, all are reported. For figures and in the text, genus and species assignments are reported when available, and when neither was available, the family taxonomic assignment was included; a unique ASV identifier is also reported. This ASV identifier can be used to look up the full taxonomic assignment from kingdom to species and the associated sequence variant in Table S2B. A phylogenetic tree was constructed in QIIME2 using the command: phylogeny align-to-tree-mafft-fasttree. The QIIME2R package was used to import QIIME2 artefacts into R (https://github.com/jbisanz/qiime2R). For RA patient samples from NYU, reads were demultiplexed using QIIME (Caporaso et al., 2010) v1.9.1 (split_libraries_fastq.py) before denoising and processing with DADA2 (Callahan et al., 2016a) v1.1.5 under MRO v3.2.5. Taxonomy was assigned using the DADA2 implementation of the RDP classifier (Wang et al., 2007) using the DADA2-formatted SILVA v128 training set (benjjneb.github.io/dada2/assign.html). Low abundance taxa were filtered out (>10 raw reads) as well as ASVs that could not be assigned taxonomy past the kingdom level. Diversity metrics were generated using Vegan (v2.5-6) and Phyloseq (v1.30.0) (McMurdie, P.J., and Holmes, S. (2013), PLoS One 8, e61217), with principal coordinate analysis (PCoA) or principal components analysis (PCA) carried out with Ape (v5.3) or Vegan, respectively. Analyses were carried out on either: (1) centered log2-ratio (clr) normalized taxonomic abundances calculated as A_{clr}=[log₂(A₁/gₐ), log₂(A₂/gₐ), . . . log₂(Aₙ/gₐ),], where A is a vector of non-zero read counts and gₐ is the geometric mean of all values of A, or (2) relative abundance calculated as proportion of reads. ANOSIM and PERMANOVA were used to detect changes in community composition using counts from rarefied data and Bray-Curtis distances. DESeq2 (v1.26.0) (Love, M. I., et al., (2014), Genome Biol 15, 550) was used to determine differentially abundant taxa on raw count data. Some ASVs were reported to have log2 fold changes that were 20 or higher. These large fold changes were found to be driven by outliers, which can sometimes occur with 16S-seq datasets. The shrinkage estimators suggested by the creators of DESeq2 were used, but found that because the calculations were driven by outliers, the log2 fold changes using these estimators were also large. The percentage of differentially abundant ASVs with log2 fold changes >20 represent a small fraction of all the significant findings (ranging from 0-34% of significant ASVs, with a median of 14%). Significance testing of longitudinal trends was determined using generalized mixed effects models using the cplm package (Sharpton et al., 2017; Zhang, 2013) (v. 0.7-7) on clr normalized values. UpSet plots were created with the UpSetR (v1.4.0) package in R (Conway, J. R., et al., (2017), Bioinformatics 33, 2938-2940). For each sample, fastq files are available in NCBI's Sequence Read Archive (SRA), accession number PRJNA656577.

Summary of 165 rRNA Amplicon Sequencing from Multiple Gnotobiotic Mouse Experiments

To assess phylum-level enrichments among differentially abundant ASVs, a. hypergeometric test was used to compare the number in each phylum among differentially abundant ASVs vs. the number present in the microbial community. The phyper function from the stats (v3.5.1) package in R was used. ANOVA was used to compare alpha diversity from multiple gnotobiotic experiments in which mice were treated with MTX. To assess changes at the phylum, genus and ASV level, a multi-factor design was carried out using DESeq2 including experiment (dose-response, individually housed, route, rescue) and MTX treatment (absence, presence) (e.g., ˜experiment+MTX). For this analysis, the low-dose samples from the dose-response experiment were excluded. The comparator groups that were used when analyzing each experiment on its own were used. For the initial dose-response experiment, high-dose was compared to PBS on day 4. Similarly, for individually housed mice, high-dose MTX was compared to PBS on day 4. For the route and rescue, day 0 was compared to day 2. These were coded as “absence” or “presence” of MTX. Multi-factor design was also used to assess changes at the phylum, genus, and ASV level on day 14 after colonization when comparing pre-MTX and post-MTX recipient mice microbiota in the FMT experiments (e.g., -donor+colonization).

qPCR for 16S Copy Number Determination

qPCR of total 16S rRNA gene copies was carried out in triplicate. Ten μL reactions with 200 nM 891F(5′-TGGAGCATGTGGTTTAATTCGA-3′ (SEQ ID NO:1))/1003R(5′-TGCGGGACTTAACCCAACA-3′(SEQ ID NO:2)) primers using a BioRad CFX384 thermocycler with iTaq™ Universal Probes Supermix (BioRad 1725132) and probe 1002P ([Cy5]CACGAGCTGACGACARCCATGCA[BHQ3]) were carried out according to the manufacturer's instructions and an annealing temperature of 60° C. Absolute quantifications were calculated against a standard curve of 8F/1542R amplified from purified bacterial DNA. Reactions were performed in triplicate and mean values were taken for further analyses. Absolute bacterial abundance was derived by adjustments for dilutions during DNA extraction, normalization, and PCR reaction preparation dividing by the total fecal mass used for DNA extraction in grams. Significance was assessed using two-way ANOVA comparing baseline and post-treatment measurements (copyNumber_Treatment*Day).

### In Vitro Bacterial Growth and Rescue Studies

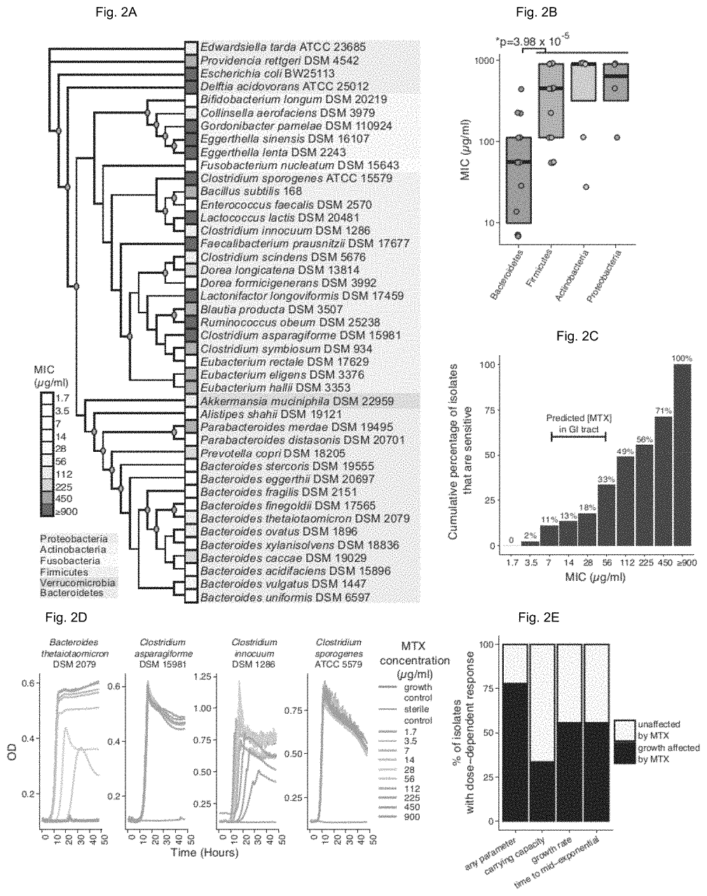

Growth parameters (carrying capacity, time to mid-exponential and growth rate) were calculated in R using GrowthCurveR (Sprouffske, K., and Wagner, A. (2016), BMC Bioinformatics 17, 1726) (v0.2.1). Curves that could not be fit by the software (i.e., generated a warning/error message) were excluded from further analysis. The fits for the remaining curves were visually assessed and removed curves with an area under the curve of <52, or estimated time to mid-exponential >48 hours which were found to be indicators of poor fit in our dataset. Determination of dose-dependency was done by fitting a linear regression with methotrexate concentration as the independent variable and the estimated growth parameter as the dependent variable using lm from the stats package (v3.5.1). For rescue studies, as above, growth parameters (carrying capacity, time to mid-exponential, growth rate, and area under the curve) were calculated in R using GrowthCurveR (Sprouffske and Wagner, 2016) (v0.2.1). Four isolates were evaluated with varying sensitivity to MTX (B. thetaiotaomicron, B. vulgatus, C. innocuum and C. symbiosum). C. asparagiforme was excluded because rescue of growth would be difficult to identify in this MTX resistant strain (FIG. 2D). Growth curves at 9 concentrations of MTX tested against 7 concentrations of folic acid were measured. Determination of dose-dependency at each tested concentration of MTX was done by fitting a linear regression with folic acid or leucovorin concentration as the independent variable and the estimated growth parameter as the dependent variable using lm from the stats package (v3.5.1). An interaction term was also included to assess whether the rescue agent demonstrated an interaction with MTX concentration (e.g., AUC˜MTX+rescue+MTX:rescue). A p-value cutoff of p<0.05 was used to identify effects that were dose-dependent based on the linear regression.

### RNA-seq Analysis

Reads were trimmed using Trimmomatic (v0.32) (Bolger, A. M., et al., (2014), Bioinformatics 30, 2114-2120), and ribosomal reads were removed using SortMeRNA (v2.1) (Kopylova, E., et al., (2012), Bioinformatics 28, 3211-3217). Reads were mapped to reference genomes using Bowtie2 (Langmead, B., and Salzberg, S. L. (2012), Nat Methods 9, 357-359) using the following options: -q, —met file, —end-to-end, —sensitive. HTSeq (v 0.8.0) was used to count the number of transcripts mapping to genes (Anders et al., 2015) using the following options: —type=CDS, —idattr:ID, —stranded=no, —minaqual=10. Differential gene expression was assessed using DESeq2 (Love et al., 2014) using the DESeqDataSetFromHTSeqCount and ddsHTSeq functions and default options. Different p_{adj} thresholds ranging from 0.01 to 0.2 were used to determine the number of differentially expressed genes, and irrespective of the threshold used, consistent percentages of each bacterial genome were affected by MTX, thus a p_{adj} threshold of 0.2 was used in subsequent analyses. EdgeR was used to identify differential gene expression (Robinson et al., 2010, Bioinformatics 26, 139-140) using the glmQLFit and glmQLFTest and the default settings, and found slightly fewer number of differentially expressed genes per isolate, but similar numbers of up-versus downregulated genes in each isolate. BlastKOALA (Kanehisa et al., 2016, J Mol Biol 428, 726-731) was used to map protein sequences from each organism to KO terms using the species_prokaryote database. KEGG pathway enrichment was carried out using clusterProfiler (Yu et al., 2012) (v3.4.1) using the enrichKEGG function. KO terms for all differentially expressed genes (both up- and downregulated with p_{adj}<0.2, DESeq) were provided and the organism parameter was set to “ko”. Heatmaps depicting enrichments were generated using the geom_tile function of ggplot2 R package (v3.3.0).

### Analysis of Untargeted Metabolomics of In Vitro Cultures

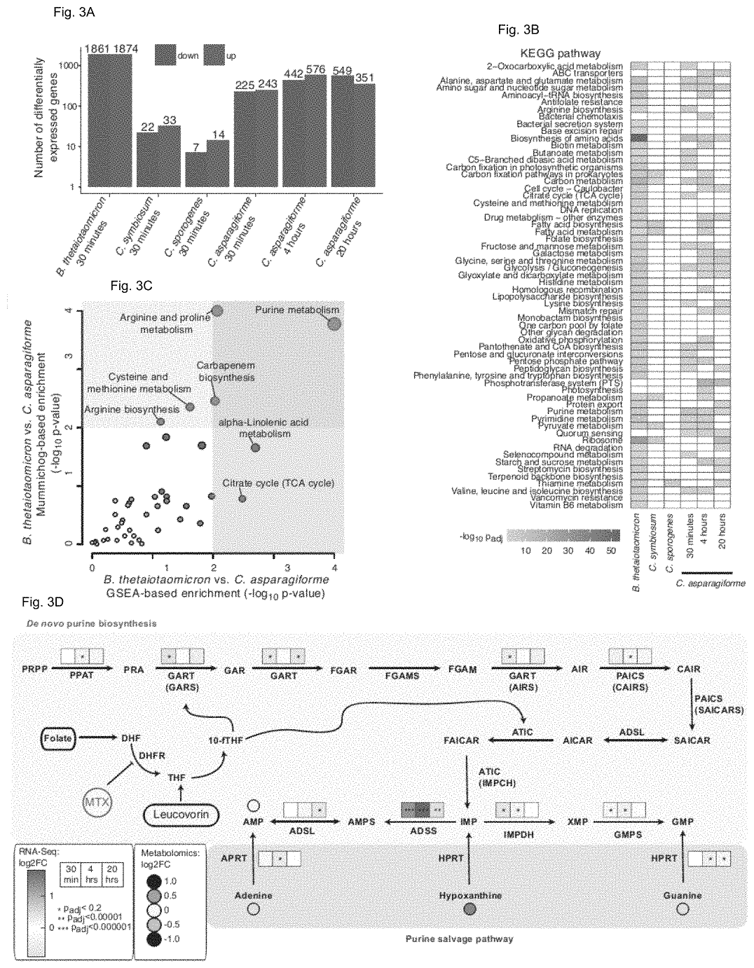

For each sample, metabolite feature areas were normalized to relative total ion counts (TIC). For FIG. 3D, multiple metabolite features mapped with high confidence to AMP, guanine, adenine, and hypoxanthine, so the TICs for these were summed in each sample. For statistical analysis, metabolite features that were differentially abundant between the sterile samples with MTX vs. DMSO were excluded from further analyses (p<0.05, Wilcoxon rank sum test). To identify metabolite features that responded differently to MTX in Bacteroides thetaiotaomicron versus Clostridium asparagiforme, peaks showing a significant interaction between bacterial isolate and MTX treatment were assessed; to do this, a two-factor ANOVA with terms for bacterial isolate, drug, and the interaction between the two was used. The BH method was used to correct for multiple testing using p.adjust in the stats package (v3.5.1) in R. Metabolite features showing a significant change with drug (drug p_{adj}<0.01) or a significant interaction between bacterial isolate and drug (interaction term, p_{adj}<0.01) were identified. Lists containing m/z values, retention times, and interaction term p-values was submitted for analysis to the MetaboAnalyst software suite “MS Peaks to Pathways” pipeline. The following parameters were set: molecular weight tolerance=10 ppm, positive mode, p-value, enforce primary ions checked, version 2.0, mummichog and GSEA checked, P-value cutoff=0.01, KEGG version current (October 2019), and Pathway library “eco_kegg.”

### Analysis of Ex Vivo Incubation of RA Patient Stool Samples

Growth curves were averaged by treatment and individual, and growth parameters were estimated using the GrowthCurveR package (v 0.2.1). Paired Student's t-tests were used to determine changes in growth parameters. Of the 30 RA patients whose baseline fecal samples were incubated with MTX in this analysis, 23 were also included in a companion paper (Artacho et al., 2020, Arthritis Rheum In press). Seven additional patients included here were excluded from the Artacho et al paper because of failure to follow-up and lack of information on clinical response to MTX.

### Shotgun Sequencing of Rheumatoid Arthritis Patient Samples

Demultiplexed reads were filtered/trimmed using fastp (Chen et al., 2018) (v0.20.0; parameters —detect_adapter_for_pe, —cut_front, —cut_tail, —cut_window_size 4, —cut_mean_quality 20, —length_required 60), aligned to the human genome (GCR38) using Bowtie2 (Langmead and Salzberg, 2012). Taxonomic profiling was done using MetaPhlAn2 (v2.7.7) (Truong et al., 2015, Nat Methods 12, 902-903) and functional profiling was done using HUMAnN2 (v0.11.1) (Franzosa et al., 2018, Nat Methods 15, 962-968) using default parameters. Of 1,136,322 gene families quantified by HUMAnN2, 168,804 were present in at least 50% of samples, and these were carried forward for further analysis. There were 5,011 KOs identified by HUMAnN2 (using the utility function humnann2_regroup_table). MaAsLin2 (v 0.99.12) (Mallick) was used to determine differential abundance of gene families and KOs using default parameters. Patient ID, clinic site of stool collection, and sequencing run were coded as random variables. Fixed variables included Month, Age, Gender, Ethnicity, and Disease Duration. KEGG pathway enrichment was carried out using clusterProfiler (Yu et al., 2012) (v3.4.1) using the enrichKEGG function. KO terms for all differentially expressed genes (both up- and down-regulated with q-value<0.25) were provided and the organism parameter was set to “ko”. For each sample, fastq files are available in NCBI's Sequence Read Archive (SRA), accession number PRJNA656577. Determination of whether a gene family was associated with purine or pyrimidine synthesis was achieved by using the pubmedR package (v 0.0.2) to search PubMed with the following query “gene family name and (purine or pyrimidine).” Results were manually curated.

### DSS Colitis Scoring

Mice were monitored for disease progression and weighed daily. Gross signs of toxicity, including hematochezia and weight loss were observed in this study. Stools were scored as follows: 0=normal stool consistency, 1=soft stool, 2=blood in stool, 3=bloody anus, 432 prolapsed anus, 5=moribund/death. Scoring was done by an observer who was blinded to the transplant group.

Analysis of Immune Cell Markers from Transplanted Gnotobiotic Mice

Quantification of cell populations was done by one of the authors (M.A.) who was blinded to the transplant group. All reported values are reported as percentage of cells or fold change percentage in which values are normalized to the pre-MTX group for each donor and treatment condition. Linear mixed effects modeling was carried out using the lme function within the nlme R package (v3.1), and donor ID was coded as a random variable. Post-MTX group was compared to the pre-MTX group in either the unchallenged or challenged mice. A heatmap of the fold change percentages in each treatment group and site was generated using the geom_tile function in the ggplot2 R package (v3.3.0).

### Correlation Analysis

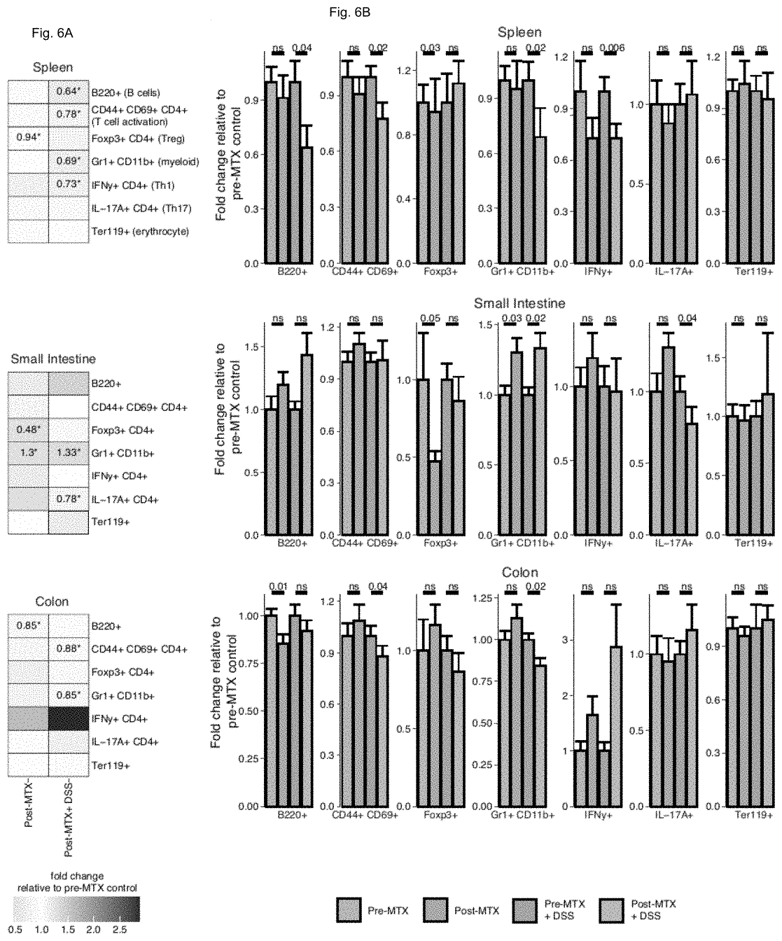

ASV abundances and immunocyte populations from a gnotobiotic experiment were correlated in which 20 mice were transplanted with pre- and post-MTX microbiota from Donor 2. Only a subset of ASVs and immunocyte populations were assessed for correlation. For ASVs, those that were previously shown to be modulated by MTX were tested. Thus, 23 ASVs that were altered by MTX in individually housed mice colonized with pre-MTX microbiota from Donor 2; 21 of the 23 ASVs were present in the communities of mice. Of these, 12 were present in at least 10 mice, and these were carried forward for assessing correlations. ASV clr-transformed abundances at day 14 were used, which represented microbial abundances prior to perturbation by DSS. For immunocyte populations, only immune markers in each organ that were differentially affected in challenged mice were tested(FIG. 6A): B220, CD44+CD69+CD4+, Gr1+CD11b+, and IFN-γ+CD4+ in the spleen; IL-17A+CD4+ and Gr1+CD11b+ in the small intestine; CD44+CD69+CD4+ and Gr1+CD11b+ and in the colon. Spearman's rank correlation is reported. Correlations that were nominally significant with p<0.05 are reported.

Provided the observations that: (i) MTX inhibits the growth of gut bacterial isolates (Kopytek, S. J., et al., Antimicrob Agents Chemother 44, 3210-3212 (2000), and Maier, L., et al., Nature 555, 623-628 (2018)); (ii) bacterial DHFR binds MTX (Bolin, J. T., et al., J Biol Chem 257, 13650-13662 (1982)); and (iii) overexpression of bacterial DHFR rescues growth in the presence of MTX (Kopytek, S. J., et al., Antimicrob Agents Chemother 44, 3210-3212 (2000)); the following examples describe for the first time that MTX acts in part by altering the gut microbiome.

### EXAMPLE 1

### Methotrexate has a broad impact on the human gut microbiota in gnotobiotic mice

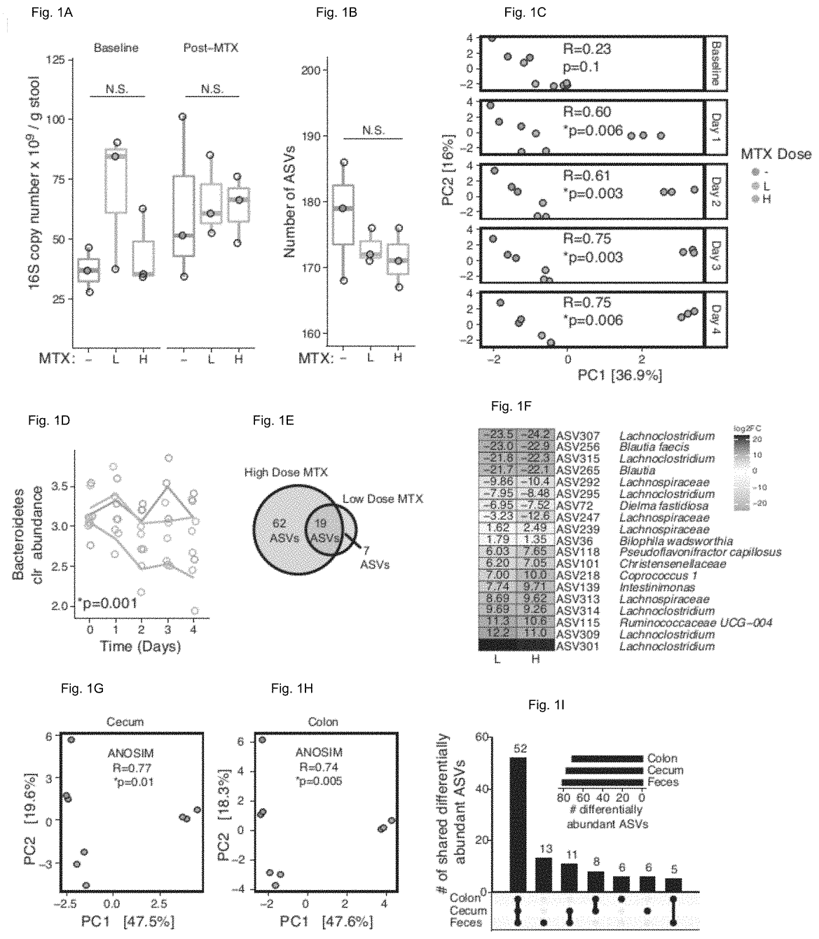

To control for the potential confounding effects of disease or prior treatment with immunomodulatory drugs, germ-free mice were colonized with a stool sample collected from a healthy human male (Donor 1) prior to the oral administration of vehicle, low-dose MTX (1 mg/kg), or high-dose MTX (50 mg/kg), selected to span doses used to treat arthritis (Koyama et al., 2017, J Pharm Pharmacol 69, 1145-1154) and cancer (Chabner and Young, 1973, J Clin Invest 52, 1804-1811) in murine models. Daily stool samples and endpoint samples from the cecum and colon were analyzed using 16S rRNA gene sequencing (16S-seq) and quantitative PCR (qPCR), and daily weights were assessed. Total colonization (FIG. 1A) and microbial richness (FIG. 1B) were comparable between groups. MTX significantly altered gut microbial community structure after 1 day (ANOSIM, R=0.60, p=0.006) and this effect persisted to the final day of treatment (ANOSIM, R=0.75, p=0.006) (FIG. 1C). There was no significant difference between groups prior to treatment (ANOSIM, R=0.23 p=0.109). High-dose MTX significantly decreased the Bacteroidetes phylum compared to vehicle controls (Day 4 DESeq p_{adj}=0.001, FIG. 1D). Analyses of longitudinal trends with a generalized linear mixed-effects model, confirmed that Bacteroidetes were decreased (slope=−0.066, p=0.007). No other phyla were significantly altered using either analysis. High-dose MTX altered 14 bacterial genera and 81 amplicon sequence variants (ASVs) spanning multiple phyla including Actinobacteria, Firmicutes, and Proteobacteria (FIG. 1E). Low-dose MTX had a more modest effect that was significant when taking into account longitudinal trends [PERMANOVA: R²=0.22, p=0.001, low-dose; R²=0.38, p=0.001, high-dose]. Low-dose MTX significantly altered 26 ASVs (FIG. 1E; Table S2C), including 19 that were also altered in a consistent direction in the high-dose group (FIG. 1F). Endpoint samples from the cecum and colon revealed significant differences in community structure (FIGS. 1G and 1H) and ASVs that were comparable to stool samples (FIG. 11).

The impact of MTX (50 mg/kg) on the gut microbiota was robust to the donor sample used, co-housing, route of delivery, and co-administration of folic acid. First, the original experimental design was replicated using individually housed mice colonized with stool from a treatment naïve RA patient (Donor 2). Microbial richness was comparable between groups (p=0.4, Wilcoxon rank sum). MTX significantly altered gut microbial community structure, with changes to the abundance of 8 genera and 23 ASVs. No phylum level changes were detected. Next, a different treatment naïve RA patient (Donor 3) was used to colonize germ-free mice prior to the oral or intraperitoneal (IP) administration of MTX (route) and prior to oral MTX with or without an equimolar level of folic acid (rescue). Neither route nor rescue resulted in a significant difference in α- or β-diversity between groups. A combined analysis of both treatment groups revealed significant shifts in the gut microbiota [Day 0 vs. 2; ANOSIM R=0.89, p=0.007 (route); ANOSIM R=0.99 p=0.002 (rescue)]. Multiple phyla were consistently affected in both experiments, along with shifts at the genus and ASV levels. Notably, consistent shifts in 41 ASVs that overlapped between route and rescue was observed.

The results of these experiments were next compared to identify common trends. Among ASVs that were differentially abundant with MTX treatment, members of the Bacteroidetes phylum were enriched in 4 out of 5 treatment groups relative to their number in the community. Other phyla did not exhibit such enrichment. To identify taxa that were similarly affected by MTX across donors and multiple experiments, data from these gnotobiotic mouse experiments was combined using multi-factor design models. As before, a-diversity was not affected by MTX (Figure S3A, p>0.05, ANOVA). Community composition was significantly affected by donor and MTX treatment. Four phyla, 31 genera, and 61 ASVs (p_{adj}<0.05, DESeq) were differentially abundant.

### EXAMPLE 2

### MTX Directly Affects Growth of Human Gut Bacteria

To test if the antimicrobial activity of MTX is sufficient to directly impact the growth of human gut bacteria or requires indirect effects driven by the host response to therapy, a panel of 45 bacterial isolates from 6 phyla was assembled, 42 of which are commonly found in the human gut microbiota. Each isolate was incubated for 48 hours with vehicle controls or MTX (1.7-900 μg/ml, 2-2,000 μM). Minimal inhibitory concentration (MIC) ranged across the full gradient, with 11 isolates resistant to the maximum concentration tested (FIG. 2A). No significant correlation was detected between growth parameters in vehicle controls and MTX sensitivity (Irhol<0.19, p>0.28). Bacteroidetes tended to be sensitive to MTX relative to the other phyla (FIG. 2B). Multiple cases where nearest neighbors had discrepant phenotypes were observed; for example, Parabacteriodes merdae vs. Parabacteroides distasonis and Lactococcus lactis vs. Clostridium innocuum (FIG. 2A).

Multiple observations support the physiological relevance of MTX for gut bacterial growth and physiology. The estimated concentration of MTX in the proximal gastrointestinal (GI) tract (22-220 μM or 10-100 μg/ml) would be sufficient to inhibit 11-33% (5-15) of the tested isolates (FIG. 2C). Furthermore, growth curves for a subset of strains revealed that 78% of the tested isolates had a least one growth parameter affected in a dose-dependent manner at sub-MIC concentrations (FIGS. 2D and 2E). In total, 36/45 (80%) isolates exhibited either full growth inhibition or alterations in growth curve parameters upon exposure to MTX at concentrations that are predicted to be found in the human gut.

### EXAMPLE 3

### Methotrexate Impacts Conserved Pathways Necessary for Cell Growth

RNA sequencing (RNA-seq) was used to identify differentially expressed transcripts in the presence or absence of MTX (100 μg/ml). 4 isolates (1 Bacteroides and 3 Clostridia) with varying sensitivity to the growth-inhibitory effects of MTX were selected. As expected, MTX induced a profound shift in gene expression in the most sensitive strain, B. theta, affecting 83% of genes in the transcriptome (FIG. 3A). In contrast, the three Clostridia tested had markedly distinct transcriptional responses that did not correlate to their sensitivity to MTX. Clostridium sporogenes and C. symbiosum exhibited a defined shift (21 and 55 genes, respectively) whereas C. asparagiforme had a robust transcriptional response (468 genes; FIG. 3A). Many of these changes were detected at more stringent p-adjusted cutoffs and with an alternative analysis method.

Next, differentially expressed metabolic pathways and modules were searched. Both purine and pyrimidine metabolism were significantly affected in C. asparagiforme and B. theta, among other pathways (FIG. 3B). While B. theta exhibited multiple pathway enrichments (57 pathways enriched among transcripts with p_{adj}<0.2), purine and pyrimidine metabolism were among the top 10 pathways (9^{th} and 5^{th}, respectively) when ranked by p-value (p<0.05 with Benjamini-Hochberg (BH) adjustment), and these enrichments were largely insensitive to the p_{adj} threshold used in our analyses. C. asparagiforme exhibited enrichment of 23 pathways, and purine and pyrimidine metabolism were 2^{nd} and 11^{th} among this list. Additionally, pathways contributing to protein synthesis, which is also known to be targeted by MTX in patients (Cronstein, 1996, Nat Rev Rheumatol 13, 41-51), were enriched in both B. theta and C. asparagiforme, such as “biosynthesis of amino acids” and more specific pathways such as “valine, leucine, and isoleucine biosynthesis” and “alanine, aspartate and glutamate metabolism” (FIG. 3B).

To assess the generalizability of these sub-MIC MTX responses to distinct growth phases, a time course experiment on the drug resistant but transcriptionally responsive C. asparagiforme was performed, comparing transcriptional profiles in response to MTX at mid- and late-exponential as well as stationary phase. Consistent with the original analyses, purine and pyrimidine metabolism continued to be among the pathways that were affected at later timepoints (FIG. 3B). Of the 41 transcripts that were differentially expressed at all three timepoints (p_{adj}<0.2, DESeq), 21 consistently changed in the same direction (5 upregulated, 16 downregulated), whereas 20 demonstrated more complicated dynamics. There were no significant differences in DHFR or AICAR transformylase expression; however, multiple genes encoding enzymes upstream and downstream of these enzymes were differentially expressed. Genes involved in de novo purine biosynthesis as well as the salvage pathway for purine synthesis showed differential expression (FIG. 3D). For example, adenylosuccinate synthase (ADSS), which is involved in converting inosine monophosphate (IMP) into adenylosuccinate (AMPS) (Pedley and Benkovic, 2017, Trends Biochem Sci 42, 141-154), was significantly upregulated along with other members of this pathway.

To gain further insights into the metabolic consequences of MTX for gut bacteria, B. theta and C. asparagiforme were subjected to untargeted metabolomics. 418 metabolic features were found that changed similarly in the two isolates and 220 that differed by species (p_{adj}<0.01, two-factor ANOVA with BH correction), and both sets showed multiple significant pathway enrichments with purine metabolism being among the most significantly enriched as assessed by two different enrichment algorithms, mummichog and GSEA (Chong et al., 2019, Curr Protoc Bioinformatics 68, e86) (FIG. 3C) . These results suggest the metabolomic response to MTX involves differential impacts on purine metabolism in these isolates. Multiple enriched pathways were consistent with our RNA-seq data (FIG. 3B), including purine metabolism, cysteine and methionine metabolism, arginine biosynthesis, and citrate (TCA) cycle.

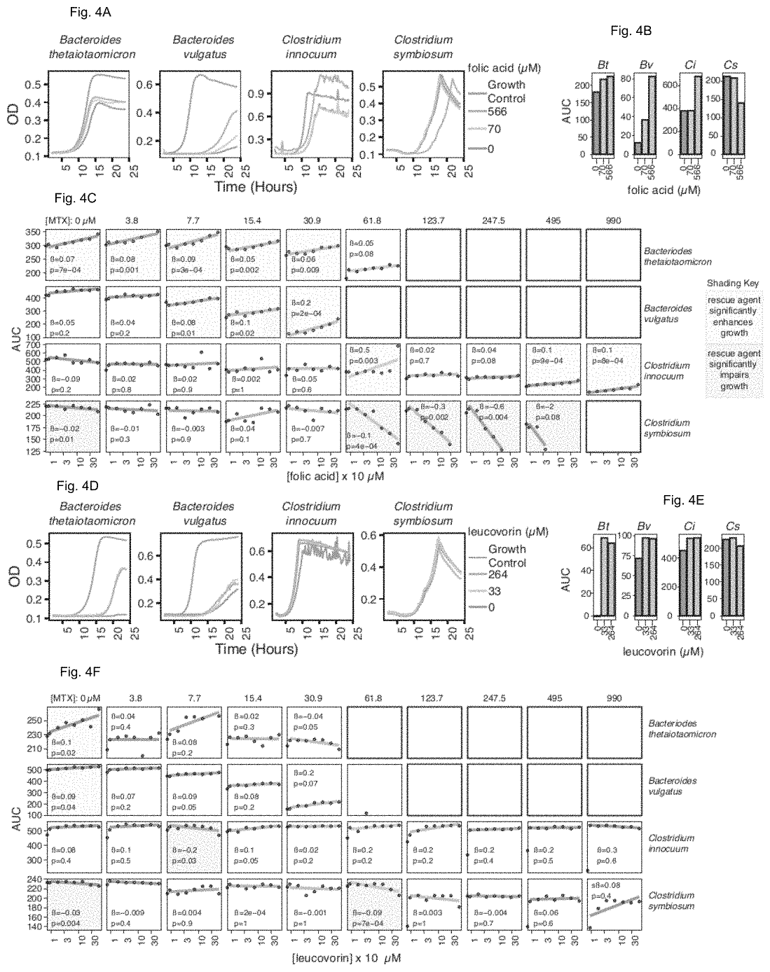

To provide functional validation of the observed transcriptomic and metabolomic changes, the ability of folic acid and leucovorin (see FIG. 3D) to rescue MTX-induced growth inhibition was quantified for four isolates of varying MTX sensitivity. B. theta growth was improved in a dose-dependent manner in response to folic acid in the presence and absence of MTX (FIGS. 4A-C). In contrast, dose-dependent increases in growth for B. vulgatus and C. innocuum were only evident in the presence of MTX (FIGS. 4A-C). Surprisingly, MTX resistant C. symbiosum was sensitized to MTX in the presence of folic acid at high doses of MTX (FIGS. 4A-C). In contrast, leucovorin improved growth of 3 of the 4 tested isolates (FIG. 4D and 4E) but did not have any significant dose-dependent effects on growth in the presence of MTX (FIG. 4F). Taken together, these findings support the hypothesis that MTX acts on bacterial DHFR (FIG. 3D).

### EXAMPLE 4

### Clinical Relevance of the Interaction Between Methotrexate and the Human Gut Microbiome

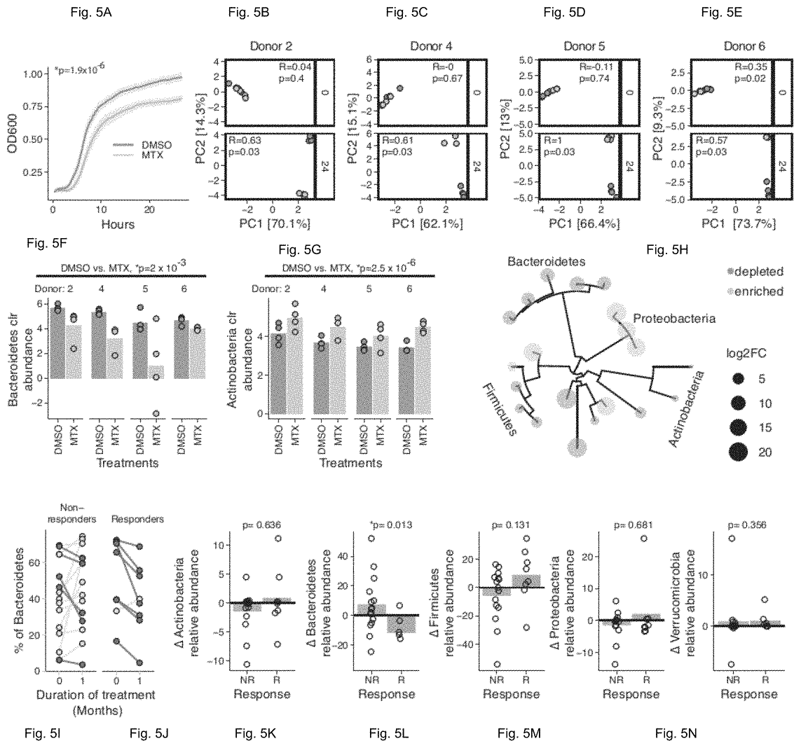

To test the impact of MTX in the context of a complex human gut microbial community stool samples obtained from 30 MTX-naive patients were incubated with MTX (100 μg/ml) or vehicle for 48 hours. MTX significantly impaired the growth of these complex microbial communities (FIG. 5A), resulting in a significant decrease in carrying capacity and a significant delay in the lag phase. 16S-seq of endpoint samples revealed a significant decrease in richness and shift in community structure (FIGS. 5B-E) in response to MTX. Consistent with the studies in mice and on isolates, Bacteroidetes decreased (FIG. 5F) and Actinobacteria increased (FIG. 5G). Significant shifts were detected in the abundance of 17 genera and 20 ASVs (FIG. 5H). This included multiple MTX-modulated ASVs seen in our in vivo experiments: B. thetaiotaomicron (ASV23), B. uniformis (ASV14), B. vulgatus (ASV26), C. aerofaciens (ASV44), and B. ovatus (ASV496), which changed in a direction similar to what was seen in vivo.

Next, whether these findings extended to RA patients by 16S-seq (n=23) and metagenomic sequencing (n=17) on longitudinal stool samples collected at baseline and 1 month after treatment initiation was considered. There were no significant differences in richness (p=0.4, Wilcoxon rank sum), community structure, or the abundance of bacterial phyla, genera, or ASVs (p_{adj}>0.05, DESeq) in pre- vs. post MTX samples across the entire cohort. There were also no significant differences in bacterial taxa abundance (ranging from phylum to species) in our metagenomic data (q-value<0.25, MaAsLin2). In contrast, analysis of gene abundance revealed a significant shift in the gut microbiome following treatment initiation: 6,409 gene families were differentially abundant over time at a nominal p-value<0.05, and 96 passed multiple testing correction (MaAsLin2 q-value<0.25, Table S7A). 334 (pₙₒₘₘₐₗ<0.05) and 5 (q<0.25) KEGG orthologs (KOs) were differentially abundant. These included gene families involved in pyrimidine synthesis (e.g., thymidylate synthase, uracil phosphoribosyltransferase) and protein synthesis (e.g., 50S ribosomal protein L22). KO enrichments were also detected for ABC transporters (p_{adj}<0.2).

Finally, whether any of the differences in the gut microbiome following treatment initiation were distinctive between MTX responders (MTX-R) and non-responders (MTX-NR) was considered. A binary classification of drug response defined as a decrease in DAS28 (Wells et al., 2009) of ≥1.8 and continued use of oral MTX without addition of other disease-modifying anti-rheumatic drugs or biologics was used. There were no significant differences in baseline disease activity parameters between MTX-R (n=8) and MTX-NR (n=15). Baseline differences in the gut microbiome between these two patient populations are the focus of another manuscript (Artacho et al., 2020). When examining changes over time, MTX-R exhibited a significant decrease in Bacteroidetes relative to MTX-NR, without any significant differences in the other phyla (FIGS. 51-N). Lower-level taxa (genus and ASV) did not significantly differ between responder groups (pₙₒₘᵢₙₐₗ>0.05, Wilcoxon rank sum), nor did taxonomic abundances (ranging from phylum to species) as assessed by shotgun sequencing (q>0.25, MaAsLin2). A significant shift was detected in 2 gene families in MTX-R, whereas MTX-NR had 508 gene families and 5 KOs with significant differences in abundance (q<0.25, MaAsLin2; Table S7A; FIG. 5O). This included gene families involved in purine and pyrimidine metabolism (e.g., thymidylate synthase and adenylosuccinate lyase; FIG. 5P), with enrichments for KOs involved in biosynthesis of secondary metabolites, 2-oxocarboxylic acid metabolism, biosynthesis of amino acids, thiamine synthesis, and folate biosynthesis among others (p_{adj}<0.2).

### EXAMPLE 5

### The Post-Treatment Microbiome Decreases Host Immune Activation

The functional impact of MTX-altered microbiota on mucosal and peripheral T cell populations in gnotobiotic mice with or without an inflammatory trigger was tested. Germ-free mice were colonized with pre- and post-treatment stool samples from the 3 MTX-Rs with the largest decrease in Bacteroidetes. Each colonization group was split into unchallenged and challenged groups, using dextran sodium sulfate (DSS) as an inflammatory trigger. In unchallenged mice, 3 immunocyte populations (including B220+ and Foxp3+CD4+ cells) were significantly lower in the post-MTX relative to pre-MTX recipients and 1 population was significantly higher (FIG. 6; range: 0.48-1.3 fold change relative to pre-MTX recipients). In challenged mice, immune infiltration and histologic colitis with DSS treatment in both groups were observed. There were no significant differences in body weight change, colitis score, or colon length between recipient groups. In challenged mice, 7 immunocyte populations (including B220+, CD44+CD69+CD4 +, and Gr+CD11b+ cells), were significantly lower and 1 was significantly higher in the post-MTX recipients (FIG. 6; range: 0.64-1.33).

To identify candidate bacterial effectors of the observed immune responses to colonization, a 16S-seq was performed on the gut microbiota of recipient mice following microbiota transplantation. Microbial community structure was distinct between donor groups, and shifts in relative abundance following colonization were observed. There were significant differences between the pre- and post-MTX recipient groups for each pair of donor samples. One phylum (Proteobacteria), 26 genera, and 41 ASVs were differentially abundant in a combined analysis of the recipients (p_{adj}<0.05, DESeq). These included ASVs that were also differentially abundant in mice or ex vivo microbial communities treated with MTX: Dielma fastidiosa (ASV72), Prevotella copri (ASV914), Eisenbergiella tayi (ASV224), Hungatella effluvia/hathewayi (ASV230), Dorea longicatena (ASV284), and Phascolarctobacterium faecium (ASV97).

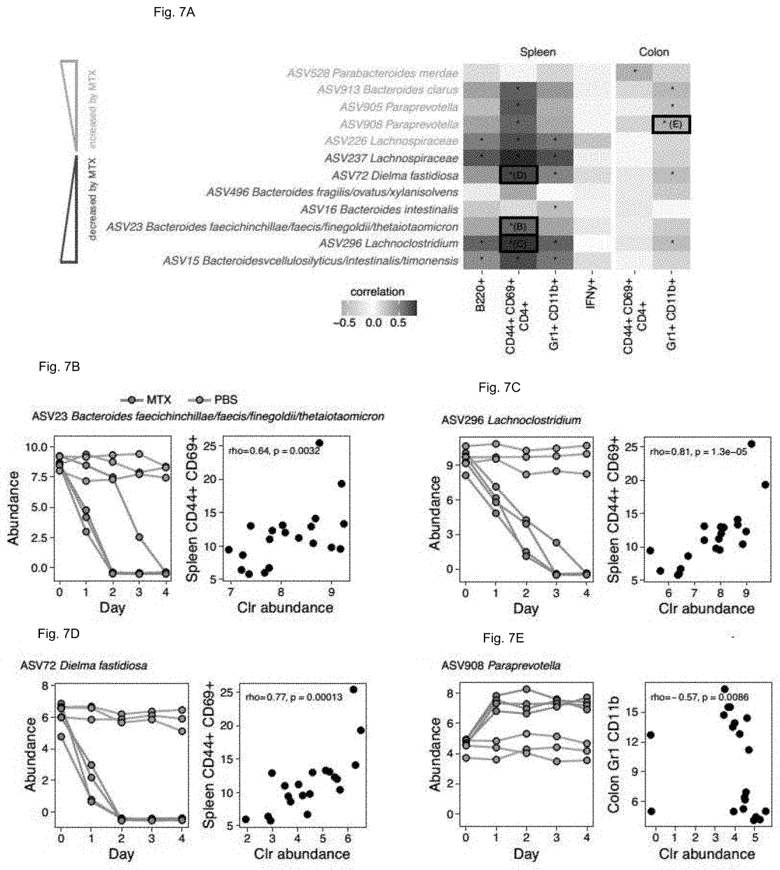

Whether MTX-modulated ASVs are associated with immune cell phenotypes was next tested. Of 23 ASVs that were shown to be modulated by MTX in a prior experiment, 12 were present in at least 10 of 20 mice in which immunocyte populations were also measured. This enabled an assessment of whether these MTX-modulated bacteria were associated with immune phenotypes. Abundances of these 12 ASVs was correlated with immune cell populations that showed differential levels in the spleen, small intestine, and colon (FIG. 7A). In the spleen, there were 18 significant positive correlations and 1 negative correlation (p<0.05, Spearman's correlation among 20 mice using day 14 abundances, FIGS. 7A-D). No significant correlations were detected between MTX-dependent ASVs and small intestinal immune cell populations. 6 ASV-immune correlations were found in the colon, all of which were negative (FIGS. 7A and 7E).

These results suggest that MTX may exert part of its anti-inflammatory effects via the gut microbiome, similar to analogous studies of the diabetes drug metformin (Wu, H., et al., Nat Med 23, 850-858 (2017), and Sun, L., et al., Nat Med 24, 1919-1929 (2018)). The post-MTX gut microbiome led to a reduction multiple cell types in both the mucosa and periphery, which included activated T cells, Th17, and IFN-γ+ T cells. These cell types are thought to play key roles in RA pathogenesis (Imboden, J. B., Annu Rev Pathol 4, 417-434 (2009)) and are consistent with evidence that MTX decreases IL-17 levels (Yue, C., et al., Rheumatol Int 30, 1553-1557 (2010)). By uncoupling the effect of MTX on the host from its effect on the gut microbiome, microbiome transplantations provide causal evidence suggesting that MTX exerts its anti-inflammatory effects in part by reducing the ability of the gut microbiome to contribute to an inflammatory response.

More broadly, the results described herein emphasize the importance of taking a broader view of toxicology that encompasses the unintended consequences of non-antibiotic drugs for our associated microbial communities. These studies demonstrate the utility of integrated studies in vitro, in gnotobiotic mice, ex vivo, and in drug naïve patients to begin to elucidate the causality and mechanism for these complex drug-microbiome-host interactions. Remarkably, the observed drug-induced changes in microbial community structure were associated with patient response, providing a potential prognostic biomarker for accelerating the stable initiation of therapy and a first step towards determining which bacterial taxa contribute to or interfere with treatment outcomes.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of treating rheumatoid arthritis (RA) in a subject comprising the steps of:
a. obtaining a first microbiome sample from the subject, wherein said subject has not received methotrexate, and determining the abundance of at least one bacterial phyla in said sample;
b. obtaining a second microbiome sample from the subject, wherein said subject has received at least one dose of methotrexate, and determining the abundance of at least one bacterial phyla in said sample;
c. comparing the abundance of step (a) with the abundance of step (b); and
d. administering methotrexate (MTX) if the abundance of step (b) is less than the abundance of step (a), or administering a medication other than MTX if the abundance of step (b) is equal to or more than the abundance of step (a).

2. The method of claim 1 wherein the abundance is measured by 16S RNA copy number per gram of sample.

3. The method of claim 1 wherein the subject is human.

4. The method of claim 1 wherein said subject received 2, 3, 4 or 5 doses of MTX prior to obtaining the second sample.

5. The method of claim 4 wherein the subject received said doses for 1, 2, 3, 4, or more weeks prior to obtaining said second sample.

6. The method of claim 1 wherein the sample is selected from the group consisting of a fecal sample, a biopsy, and a noninvasive capsule endoscopy sample.

7. The method of claim 1 wherein the abundance of 2, 3, 4, 5 or more phyla of bacteria is determined.

8. The method of claim 1 wherein the bacterial phyla is selected from the group consisting of Bacteroidetes, Firmicutes, Actinobacteria, Proteobacteria, Fusobacteria and Verrucomicrobia.

9. The method of claim 8 wherein the phylum is Bacteroidetes.

10. The method of claim 8 wherein the bacterial phyla comprises:
(a) one or more members of a class selected from the group consisting of Negativicutes, Bacteroidia, Clostridia, Betaproteobacteria, Erysipelotrichia, Actinobacteria, Deltaproteobacteria, Verrucomicrobiae, Coriobacteriia, and Bacilli;
(b) one or more members of an order selected from the group consisting of Selenomonadales, Bacteroidales, Clostridiales, Erysipelotrichales, Coriobacteriales, Desulfovibrionales, Verrucomicrobiales, Burkholderiales, Lactobacillales, and Bifidobacteriales;
(c) one or more members of a family selected from the group consisting of Acidaminococcaceae, Porphyromonadaceae, Lachnospiraceae, Ruminococcaceae, Bacteroidaceae, Eubacteriaceae, Erysipelotrichaceae, Coriobacteriaceae, Desulfovibrionaceae, Clostridiales Incertae Sedis XIII, Clostridiaceae Prevotellaceae, Verrucomicrobiaceae, Sutterellaceae, Defluviitaleaceae, Streptococcaceae, Bifidobacteriaceae, Clostridiaceae, and Leuconostocaceae;
(d) one or more members of a genus selected from the group consisting of Phascolarctobacterium, Barnesiella, Dorea, Blautia, Clostridium XlVa, Clostridium XlVb, Anaerorhabdus, Eubacterium, Bacteroides, Eggerthia, Collinsella, Flavonifractor, Ruminococcus, Gordonibacter, Bilophila, Anaerofustis, Mogibacterium, Coprococcus, Oscillibacter, Clostridium sensu stricto, Lachnospiraceae UCG-008, Lachnoclostridium, Eubacterium fissicatena group, Dielma, Marvinbryantia, Subdoligranulum, Lachnospiraceae NK3A20 group, Prevotella, Akkermansia, Odoribacter, Clostridium XVIII, Ruminococcus2, Parasutterella, Clostridium IV, Defluviitalea, Streptococcus, Bifidobacterium, Butyricicoccus, Clostridium sensu stricto, and Weissella; and
(e) one or more members of a species selected from the group consisting of Phascolarctobacterium faecium, Blautia hansenii/producta, Clostridium XlVa bolteae/clostridioforme, Clostridium XlVa scindens, Bacteroides uniformis, Clostridium XlVa oroticum, Bacteroides ovatus, Bacteroides caccae, Bacteroides cellulosilyticus/timonensis, Bacteroides intestinalis, Collinsella aerofaciens, Blautia faecis, Anaerofustis stercorihominis, Clostridium sensu stricto celatum/disporicum, Lachnospiraceae UCG-008 uncultured organism, Bacteroides thetaiotaomicron, Marvinbryantia metagenome, Lachnospiraceae NK3A20 group uncultured bacterium, Akkermansia muciniphila, Bacteroides dorei/vulgatus, Bacteroides massiliensis, Coprococcus comes, Blautia obeum, Bacteroides faecichinchillae/faecis/thetaiotaomicron, Clostridium XlVa glycyrrhizinilyticum, Parasutterella excrementihominis, Streptococcus anginosus subsp. anginosus, Bacteroides uniformis, Bacteroides thetaiotaomicron, and Butyricicoccus uncultured bacterium.

11. The method of claim 1 wherein the MTX is selected from the group consisting of RASUVO®, TREXALL®, OTREXUP®, Methotrexate LPF Sodium, XATMEP®, JAMP-Methotrexate, METOJECT® and PMS-Methotrexate.

12. The method of claim 11 wherein the MTX is administered by a route selected from the group consisting of subcutaneous injection, intravenous injection, and oral tablet.

13. The method of claim 1 wherein the medication is selected from the group consisting of nonsteroidal anti-inflammatory drugs (NSAIDs), steroids, disease-modifying antirheumatic drugs (DMARDs), and biological agents.

14. The method of claim 13 wherein the NSAID is selected from the group consisting of ibuprofen and naproxen sodium.

15. The method of claim 13 wherein the steroid is selected from the group consisting of prednisone, prednisolone, methylprednisolone, dexamethasone, hydrocortisone, triamcinolone, and betamethasone.

16. The method of claim 13 wherein the DMARD is selected from the group consisting of leflunomide (Arava), hydroxychloroquine (PLAQUENIL®), sulfasalazine (AZULFIDINE®), azathioprine (IMURAN®) and minocycline.

17. The method of claim 13 wherein the biological agent is selected from the group consisting of abatacept (ORENCIA®), adalimumab (HUMIRA®), anakinra (KINERET®), baricitinib (OLUMIANT®), certolizumab (CIMZIA®), etanercept (ENBREL®), golimumab (SIMPONI®), infliximab (REMICADE®), rituximab (RITUXAN®), sarilumab (KEVZARA®), tocilizumab (ACTEMRA®), tofacitinib (XELJANZ®), and upadacitinib (RINVOQ®).

18. A method of reducing inflammation in the gut and/or joints of a subject comprising the steps of
a. obtaining a sample from the subject;
b. determining the abundance of at least one bacterial phylum in the sample
c. comparing the abundance of said at least one bacterial phylum to a threshold amount; and
d. administering methotrexate (MTX) if the abundance of said at least one bacterial phylum is less than said threshold amount, or administering a medication other than MTX if the abundance of said at least one bacterial phylum is more than said threshold amount.

19. The method of claim 18 wherein said subject is suffering from RA and/or inflammatory bowel disease (IBD).

20. The method of claim 19 wherein the IBD is selected from the group consisting of Crohn's disease or ulcerative colitis.

21. A method of treating rheumatoid arthritis (RA) in a subject comprising the steps of:
a. obtaining a microbiome sample from the subject;
b. determining the abundance of at least one bacterial phyla in the sample;
c. comparing the abundance of said at least one bacterial phyla to a threshold amount; and
d. administering methotrexate (MTX) if the abundance of said at least one bacterial phyla is less than said threshold amount, or administering a medication other than MTX if the abundance of said at least one bacterial phyla is more than said threshold amount.
